SEQUENCE LISTING (3 sequences)

SEQ ID NO: 1 - protein, 654 aa, Artificial Sequence
  Synthetic Sequence
SDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLR

SEQ ID NO: 2 - protein, 1242 aa, Artificial Sequence
  Synthetic sequence
SDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 421 aa, Artificial Sequence
  synthetic sequence
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQ

(19) United States
(12) Patent Application Publication
(10) Pub. No.: US 2022/0034896 A1
(43) Pub. Date: Feb. 3, 2022

(54) METHODS FOR QUANTITATIVE ANALYSIS OF ONE OR MORE BIOMARKERS

(71) Applicant: The Research Foundation for the State University of New York, Albany, NY (US)

(72) Inventor: Nathaniel Cady, Delmar, NY (US)

(21) Appl. No.: 17/368,711

(22) Filed: Jul. 6, 2021

Related U.S. Application Data
(60) Provisional application No. 63/048,001, filed on Jul. 3, 2020.
(60) Provisional application No. 63/215,839, filed on Jun. 28, 2021.

(51) Int. Cl. G01N 33/58

(52) U.S. Cl. CPC G01N 33/582; G01N 2800/26

(57) ABSTRACT

The present disclosure relates to apparatuses and methods for detecting the amount and/or type of one or more analytes-of-interests such as biomarkers in a sample. In embodiments, the disclosure includes a method for determining a humoral response due to the presence of a target infectious agent or vaccine. In embodiments, detecting emission light from one or more fluorescent complexes is used to determine a type and/or quantity of the plurality of biomarkers.

### CROSS-REFERENCE TO RELATED APPLICATIONS

The present disclosure claims priority or the benefit under 35 U.S.C. § 119 of U.S. to provisional application Nos. 63/048,001 filed Jul. 3, 2020, and 63/215,839 filed 28 Jun. 2021, and U.S. application Ser. No. 17/367,141 filed 2 Jul. 2021, all of which are fully incorporated herein by reference.

### REFERENCE TO A SEQUENCE LISTING

This application contains a Sequence Listing in computer readable form, which is incorporated herein by reference.

### FIELD OF THE INVENTION

The present disclosure relates to apparatuses and methods for detecting the amount and/or type of one or more analytes-of-interests such as biomarkers in a sample. Further, the present invention relates to the fields of immunology and virology, including methods of detecting antibody formation and/or determining viral or vaccine induced immunity in a subject. In embodiments, the present disclosure relates to detecting a humoral response in a subject in response to an infectious agent or vaccine directed against an infectious agent. In embodiments, the present disclosure relates to apparatuses and methods for the quantitative detection of biomarkers against an infectious agent or vaccine, such as detecting one or more human antibodies against the SARS-CoV-2 virus, viral variants of SARS-CoV-2, and/or one or more vaccines directed against SARS-CoV-2, and variants of SARS-CoV-2.

### BACKGROUND

Infectious diseases cause acute and chronic health problems in humans, whether due to pathogens such as bacteria, fungi, viruses, or other factors. Infections occur when pathogens enter the body and multiply. Disease occurs when the cells of the body are damaged by the infection, and signs and symptoms of illness appear.

In response to a foreign substance, in particular to an infectious agent (such as bacteria, viruses, fungi, and parasites), a subject may trigger an immune response. The humoral response corresponds to the production of immunoglobulins (antibodies). These immunoglobulins diffuse into the subject's blood, tissues, or mucous membranes and allow the subject to defend itself against the infectious agent. Such a defense may be in the form of a capacity to neutralize or to prevent reproduction of the infectious agent or a new infection by the infectious agent.

Infectious diseases continue to develop naturally resulting from new pathogens and strains infecting human populations. For example, in the mid-1970's Lyme disease caused by ticks infected with the spirochete bacteria, Borrelia burgdorferi, was identified in rural Connecticut causing symptoms similar to rheumatoid arthritis. More recently the World Health Organization (WHO) declared infection by the novel Severe Acute Respiratory Syndrome Coronavirus 2 (SARS-CoV-2), as a pandemic, and termed the related disease as coronavirus disease 2019 (COVID-19). Variants of interest of SARS-CoV-2 and variants of concern of SARS-CoV-2 have been identified, and it is expected that additional variants will present as the virus continues to evolve over time.

Upon infection, the human body senses foreign substances or antigens and the immune system works to recognize antigens and rid the body of the antigens. Adaptive immunity develops when individuals are exposed to antigens and typically becomes more prominent after several days of infection, as antigen-specific T and B cells have undergone clonal expansion. For a general overview of the human immune system response, see for example, Chaplin, Overview of the Immune Response, J Allergy Clin Immunol. 2010 February; 125(2 Suppl 2): S3-23 (herein entirely incorporated by reference). A typical immune response may include the production of biomarkers such as immunological agents or immunoglobulins in various isoforms including IgG, IgA, IgM and IgE antibodies, and by somatic mutations in the antigen-binding domains of the heavy and light chains of these antibodies.

The inventor has observed the quantity and type of biomarkers such as immunological agents may vary over the course of infection, disease, and recovery, and that biomarkers may be monitored to determine a subject's status. Such monitoring may be useful for disease intervention or managing a treatment strategy. Further, monitoring biomarkers such as antibodies throughout a subject's recovery period is problematic in that it typically requires multiple pieces of laboratory equipment in a complex laboratory environment to quantify and type the biomarkers, especially where the biomarkers may be present at low concentrations or where sample quality is poor. Moreover, the form of the biological samples may present problems depending upon whether the sample is serum, whole blood, or dried blood, as varying conditions of the sample may make evaluation thereof difficult.

Further, the inventor has found that detection of a virus-of-interest alone has several shortcomings in treatment management. For example, one currently available diagnostic test, a PCR based approach for COVID-19 (SARS-CoV-2) testing has several deficiencies, namely the test only detects the presence of virus in the sample and does not determine the immune status of one or more subjects, especially where subjects have different immune responses to an infection.

The inventor has also observed that medical doctors or clinicians would benefit from monitoring a subject's status and the immune response over the duration of an illness or recovery period.

Accordingly, there is a need for improved methods, apparatuses, and assays for the detection and identification of one or more biomarkers such as those made in response to viral diseases such as COVID-19 caused by SARS-CoV-2, bacterial diseases, or other diseases. What is needed are methods of simultaneously detecting and/or quantifying antibodies against an infectious agent or vaccine, such as detecting one or more human antibodies against the SARS-CoV-2 virus, viral variants of SARS-CoV-2, and/or one or more vaccines directed against SARS-CoV-2, and variants of SARS-CoV-2. Moreover, what is also needed are methods of surveilling a subject's immune status to estimate or determine antibody generation in response to infection or vaccination.

### SUMMARY

The present disclosure includes methods of detecting antibody formation and/or determining viral or vaccine induced immunity in a subject. In embodiments, the present disclosure relates to detecting a humoral response in a subject in response to an infectious agent or vaccine directed against an infectious agent. In embodiments, the present disclosure relates to apparatuses and methods for the quantitative detection of biomarkers against an infectious agent or vaccine, such as detecting one or more human antibodies against the SARS-CoV-2 virus, viral variants of SARS-CoV-2, and/or one or more vaccines directed against SARS-CoV-2, and variants of SARS-CoV-2.

In embodiments, the present disclosure includes a method for determining a humoral response due to the presence of a target infectious agent or vaccine, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

In embodiments, the present disclosure includes a method for determining a humoral response due to the presence of a target infectious agent or vaccine, including: capturing one or more biomarkers on a substrate configured to bind a plurality of biomarkers to a plurality of binding sites, wherein one or more binding sites bind one or more biomarkers, and wherein when one or more biomarkers are present, forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the biomarkers include immunoglobulins including one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.

In embodiments, the present disclosure includes an apparatus for quantitative analysis of a plurality of biomarkers contained in a sample, including: a substrate including a plurality of binding sites, wherein two or more of the plurality of binding sites include two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes; and a grating-coupled fluorescent plasmonic (GC-FP) detection platform, wherein the platform is configured for contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes, wherein the two- or more biomarkers-of-interest are selected from immunoglobulins including one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.

In some embodiments, the present disclosure includes a method for determining a humoral response due to the presence of a target infectious agent or vaccine, including: detecting in a biological sample of a subject at least one target antibody that is susceptible of being produced by the subject when the subject is vaccinated or infected by a target infectious agent, wherein the method includes: capturing the at least one target antibody on a substrate configured to bind the at least one target antibody to a plurality of binding sites, wherein at least two binding sites are configured to bind at least two target antibodies, and wherein when one or more target antibodies are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the at least one target antibody, wherein the at least one target antibody is selected from immunoglobulins comprising one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.

In embodiments, the present disclosure includes a method of detecting antibodies to a human disease, including: vaccinating a subject with a vaccine to generate one or more antibodies to a predetermined infectious agent or variant thereof; capturing one or more antibodies on a substrate configured to bind the one or more antibodies to a plurality of binding sites, wherein at least two binding sites are configured to bind at least two antibodies, and wherein when one or more antibodies are present forming one or more bound antibodies-of-interest; contacting the one or more bound antibodies-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a quantity of the one or more antibodies.

In embodiments, the present disclosure includes a method of measuring antibody levels that resulted from COVID-19 infection or vaccination against SARS-CoV-2 and variants thereof, including: (a) contacting a biological sample to a microchip-based grating coupled fluorescent plasmonic (GC-FP) assay; and (b) determining a quantity of antibodies directed against SARS-CoV-2, variants of SARS-CoV-2, or a vaccine directed against SARS-CoV-2. In embodiments, the method may include vaccinating a subject in need thereof with a vaccine directed against SARS-CoV-2, or a variant thereof.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a flowchart of a method for a simultaneous qualitative and quantitative analysis of a plurality of biomarkers in accordance with some embodiments of the present disclosure.

FIGS. 2A-2D are illustrative cross-sectional views of the substrate during different stages of the process sequence of FIG. 1 in accordance with some embodiments of the present disclosure.

FIG. 3 depicts a tool suitable to perform methods for processing a substrate in accordance with some embodiments of the present disclosure.

FIG. 4 is a photograph of a microfluidic flow cell such as GC-FP biochip suitable for use with some embodiments of the present disclosure.

FIG. 5 is a photograph of a fluorescent image in accordance with the present disclosure.

FIG. 6A is a map of antigen spots in accordance with the present disclosure.

FIG. 6B is an image of antigen spots of FIG. 6A processed in accordance with the present disclosure.

FIG. 7A is a plot of GC-FP intensity in accordance with one embodiment of the present disclosure.

FIG. 7B is a plot showing the scoring of the plot shown in FIG. 7A.

FIGS. 8A and 8B are photographs of a dipstick and holder in accordance with embodiments of the present disclosure.

FIG. 9 is a photograph of six 4×4 GC-FP chips with 3×3 grid of binding spots in accordance with the present disclosure.

FIG. 10 shows an image of serum and blood test using the kit shown in FIG. 9.

FIGS. 11A and 11B show graphs and images of dried blood samples analyzed in accordance with the present disclosure.

FIG. 12 show graphs and images of samples analyzed in accordance with the present disclosure.

FIG. 13 relates to data collected from samples analyzed in accordance with the present disclosure.

FIGS. 14A and 14B depict Human IgG levels against SARS CoV2 antigens, and Human IgG levels against SARS-CoV2 antigens throughout the vaccination sequence. More specifically, FIG. 14A depicts Human IgG levels against SARS CoV2 antigens, measured by GC-FP, for vaccinated individuals vs. unvaccinated individuals. The Mann-Whitney test and ROC analysis (FIG. 20B) were used to determine statistical significance.

FIG. 14B depicts Human IgG levels against SARS CoV2 antigens throughout the vaccination sequence (Pfizer-BioNTech) for 10 different subjects, collected pre-vaccination, at the time of the 2^{nd} dose of vaccine, and 2 weeks after the 2^{nd} dose of vaccine. One-way ANOVA followed by Dunnett's multiple comparison testing was performed (*p=0.03, ** p=0.002, *** p=0.0002, **** p<0.0001).

FIG. 15 depicts human IgG levels for a single individual over the course of vaccination with the Pfizer-BioNTech vaccine, measured with GC-FP.

FIG. 16 depicts IgG levels against SARS CoV2 antigens for uninfected, previously infected, and vaccinated individuals. Uninfected samples were collected prior to vaccination, from individuals who reported no prior COVID-19 symptoms, and tested negative via PCR and/or antibody testing (n=42). Other samples were from PCR confirmed COVID-19 positive subjects who were hospitalized (n=3), PCR confirmed COVID-19 positive subjects who were not hospitalized CoV2 (n=5), and previously COVID-19 positive subjects who received subsequent vaccination (n=9). Samples were also collected from subjects who were at least 2 weeks past full vaccination with Pfizer-BioNTech (n=17), Moderna (n=8), or 2 weeks after receiving the Johnson & Johnson vaccine (n=9). One-way ANOVA followed by Dunnett's multiple comparison testing was performed (*p=0.03, ** p=0.002, *** p=0.0002, **** p<0.0001).

FIG. 17 depicts fold-difference in antibody levels against antigens from SARS CoV2 variant strains B.1.1.7 and B.1.351 vs. antigens from the original 2019 SARS CoV2 strain. Samples included those from individuals who were hospitalized (n=3); non-hospitalized CoV2 positive (n=5); previously CoV2 positive with subsequent vaccination (n=9); and at least 2 weeks past vaccination with Pfizer-BioNTech (n=17), Moderna (n=8), or Johnson & Johnson vaccine (n=9). One-way ANOVA followed by Dunnett's multiple comparison testing was performed (*p=0.03, ** p=0.002, *** p=0.0002, ****p<0.0001).

FIGS. 18A-18F depicts various plots of the present disclosure. FIGS. 18A and 18B depict IgG levels from dried blood spots measured by GC-FP diagnostic ratio compared to competitive ELISA by eluate from the same dried blood spot samples. Both GC-FP and ACE2 competitive binding were performed for RBD antigen from the original 2019 SARS CoV2 and the variant strains B.1.1.7 and B.1.351. Testing was performed with dried blood spots collected from vaccinated subjects (3 Pfizer-BioNTech, 3 Moderna, 2 Johnson & Johnson) and subjects who were both previously infected and then vaccinated with Pfizer-BioNTech or Moderna vaccines (n=3). One-way ANOVA followed by Dunnett's multiple comparison testing was performed (*p=0.03, ** p=0.002, *** p=0.0002, **** p<0.0001). Percent ACE2 binding inhibition from competitive ELISA assay is shown for a dried blood spot sample (FIG. 18C) from a Pfizer-BioNTech vaccinated subject and blood serum from a hospitalized, COVID-positive subject (FIG. 18D). Vertical dotted lines represent the dilution factor used in the corresponding GC-FP test for each sample. Fold difference in binding inhibition and fold difference in GC-FP diagnostic ratio was plotted for variant antigens (RBD B.1.1.7 and RBD B.1.351) vs. RBD 2019 CoV2 (FIGS. 18E & 18F).

FIG. 19 depicts a top-down view of a GC-FP SARS CoV2 antigen chip of the present disclosure, and a table depicting the antigen and spot number on the chip.

FIGS. 20A and 20B depict various plots in accordance with the present disclosure. FIG. 20A depicts Human IgG levels against SARS CoV2 antigens, measured by GC-FP, for vaccinated individuals vs. unvaccinated individuals. The Mann-Whitney test and ROC analysis (see supplementary figure S2) were used to determine statistical significance (*p=0.03, ** p=0.002, *** p=0.0002, **** p<0.0001). FIG. 20B depicts ROC analysis of human IgG levels (measured by GC-FP) against 2019 SARS CoV2 for vaccinated vs. unvaccinated individuals. Area under the curve (AUC) and the GC-FP diagnostic ratio threshold are reported. The GC-FP diagnostic ratio threshold needed to achieve 100% specificity (false positive rate) is reported, as well as the sensitivity (true positive rate) that can be achieved while maintaining 100% specificity.

FIG. 21 depicts various plots of the present disclosure. FIG. 21 depicts Human IgG levels against SARS CoV2 antigens from the original 2019 SARS CoV2 strain and variant strains B.1.1.7 and B.1.351. GC-FP diagnostic ratio is reported as a measure of IgG level against each antigen, for multiple exposure scenarios and vaccination status (hospitalized and non-hospitalized CoV2 positive, previously CoV2 positive with subsequent vaccination, and at least 2 weeks past vaccination with Pfizer-BioNTech, Moderna or Johnson & Johnson vaccine). One-way ANOVA followed by Dunnett's multiple comparison testing was performed (*p=0.03, ** p=0.002, *** p=0.0002, **** p<0.0001).

FIG. 22 is a plot depicting a correlation of the fold change in ACE2 binding inhibition or GC-FP diagnostic ratio for B.1.1.7 and B.1.351 variants of RBD vs. 2019 CoV2 RBD.

FIGS. 23A and 23B depict a table depicting the antigen and spot number on a chip including a plurality of antigens to a plurality of SARS-CoV-2 variants, and a top-down view of an antigen chip of the present disclosure, respectively.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The apparatuses and methods described herein relate to detecting and/or quantifying biomarkers-of-interest, such as from infected or diseased subjects. In embodiments, apparatuses and methods for detecting and/or quantifying biomarkers in a specimen, such as from whole blood, blood serum, or dried blood are described.

In embodiments, the present disclosure relates to a method for a simultaneous qualitative and quantitative analysis of a plurality of biomarkers contained in a sample, including: capturing one or more biomarkers on a substrate configured to bind a plurality of biomarkers to a plurality of binding sites, wherein one or more binding sites, or in embodiments, at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

Embodiments of present disclosure advantageously provide: improved methods, apparatuses, and assays for the detection and identification of one or more biomarkers such as those made in response to viral diseases, bacterial diseases or other diseases; methods that do not require complex laboratory infrastructure and can be applied to any infectious disease that elicits an immune response, or where antibody levels need to be determined for diagnosis/prognosis; methods and apparatuses for detecting and/or simultaneously quantifying biomarkers such as antibodies over the course of treatment, such as from a blood, blood serum, or dried blood; or methods of surveilling a subject's immune status to estimate or determine type of immunological response to an infection. Additional benefits of the methods and apparatuses of the present disclosure may include providing a comprehensive diagnostic strategy that in a single test allows for simultaneous detection of antibodies; cytokines; and other biomarkers essential in properly detecting pathogen infection such as from the bacteria or virus such as SARS-CoV-2 and/or variants of SARS-CoV-2 and the severity of infection in each subject. Advantages may be especially important where convalescent subjects such as COVID-19 patients are being screened for use of their serum/blood sample to provide to infected patients for therapy, or where subjects desire or need an immune status.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the term “ACE2” refers to Angiotensin II converting enzyme (ACE2, EC 3.4.17.23). ACE2 is a protein that catalyzes the cleavage of angiotensin I into angiotensin 1-9, and angiotensin II into the vasodilator angiotensin 1-7. In addition, the encoded protein is a functional receptor for the spike glycoprotein of the human coronavirus HCoV-NL63 and the human severe acute respiratory syndrome coronaviruses, SARS-CoV and SARS-CoV-2 (COVID-19 virus). ACE2 converting enzyme activity may be measured using, inter alia, fluorometric activity assay kits and by other known methods such as those described in Measurement of Angiotensin Converting Enzyme 2 Activity in Biological Fluid (ACE2), Methods Mol Biol. 2017; 1527:101-115.

The term “antibody” as used herein refers to an immunoglobulin molecule capable of specific binding to a target antigen or biomarker, such as a carbohydrate, polynucleotide, lipid, polypeptide, peptide etc., via at least one antigen recognition site (also referred to as a binding site), located in the variable region of the immunoglobulin molecule.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

As used herein, the terms “bind” and “binding” generally refer to the non-covalent interaction between a pair of partner molecules or portions thereof (e.g., antigenic protein-binding partner complexes) that exhibit mutual affinity or binding capacity. In embodiments, binding can occur such that the partners are able to interact with each other to a substantially higher degree than with other, similar substances. This specificity can result in stable complexes (e.g., antigenic protein-binding partner complexes or bound biomarkers-of-interest) that remain bound during handling steps such as chromatography, centrifugation, filtration, and other techniques typically used for separations and other processes. In embodiments, the interaction between a target region of an antigenic protein and a binding partner that binds specifically thereto is a non-covalent interaction. In some instances, the interaction between a binding partner and a non-target region of an antigenic protein is a non-covalent interaction. However, in other instances, the interaction between a binding partner and a non-target region of an antigenic protein may be a covalent interaction. In embodiments, a protein complex comprising the antigenic protein and the binding partner may be contacted with a chemical crosslinking reagent that causes covalent bonds between the antigenic protein and the binding partner to be formed. In another example, the antigenic protein may contain a first reactive chemical moiety (handle) and the one or more binding partners may each contain a second reactive chemical moiety (handle), wherein the first and second chemical reactive moieties can react with each other to form a covalent bond. Exemplary reactive chemical moieties include those useable in “click” chemistry, which is a class of biocompatible small molecule reactions commonly used in bioconjugation, allowing the joining of substrates of choice with specific biomolecules. Click chemistry is not a single specific reaction, but refers to a way of generating products that follow examples in nature, which also generates substances by joining small modular units. In one example, the antigenic protein may have a first reactive chemical moiety such as a clickable handle like an azide, and the binding partner(s) could have a complementary reactive handle such as, for example a strained cyclooctyne, or vice versa. When these reactive chemical moieties come into proximity when the antigenic protein and the one or binding partners interact to form a protein complex, they can react with each other to form a covalently bond between the proteins.

“Biological sample” refers to isolation of tissue and/or fluid from a subject that is being tested for a disease state. In embodiments, any biological sample can be used by the present disclosure, provided the sample may contain, or contains the biomarkers for the disease state being tested, such as blood, blood components, urine, saliva or breath. In embodiments, biological samples include blood, blood plasma, or dried blood.

“Biomarker” refers to biological compounds that are involved in one or more biological pathways that are associated with a disease state. Accordingly, for infections, the biomarker can be involved with pathways that regulate the host immune response. A “profile” of biomarkers or “biomarker profile” refers to the amount or concentration of two or more biomarkers. Such a profile provides useful top-level “fluxomics” information about whether certain types or pools of biomarkers are elevated or depleted. A disease state can have a specific biomarker profile, and more particularly a time-dependent biomarker profile. Accordingly, the biomarker profile is also referred to as a disease state “finger-print” that permits the identification of a disease state based on a measured biomarker profile. A biomarker that is “related to the disease state” refers to biomarker profiles that change depending on the disease state and provides a means for assessing a subject's disease state based on the measured biomarker levels.

The terms “elute” and “eluting” refer to the disruption of non-covalent interactions between partner molecules (e.g., a modified antigenic protein and a binding partner) such that the partners become unbound from one another. The disruption can be effected via introduction of a competitive binding species, or via a change in environmental conditions (e.g., ionic strength, pH, or other conditions).

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

As used herein the terms “dye” and “label” are used interchangeably to designate fluorescent molecules.

The term “fluorescent complexes” as used herein means complexes such as biomolecules, proteins, protein conjugates and the like that can fluoresce.

The term “fluoresce” as used herein means to exhibit or undergo the phenomenon of fluorescence.

The term “fluorescence” as used herein means the emission of light by a substance that has absorbed light or other electromagnetic radiation of a different wavelength.

The term “fluoresced light” as used herein means emitted light from fluorescence of a substance. In most cases, emitted light has a longer wavelength, and therefore lower energy, than the absorbed radiation. However, when the absorbed electromagnetic radiation is intense, it is possible for one electron to absorb two photons; this two-photon absorption can lead to emission of radiation having a shorter wavelength than the absorbed radiation.

The term “isolated” refers to a chemical or biomolecule species such as a protein, protein complex, or DNA sequence that is removed from at least one component with which it is naturally associated.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used here, the term “SARS-CoV-2” refers to virus classified within the genus Betacoronavirus (subgenus Sarbecovirus) in the family Coronaviridae (subfamily Orthocoronavirinae), a family of single-strand positive-sense RNA viruses. In embodiments, the term “SARS-CoV-2” includes variants of SARS-CoV-2.

The term “solid support” is used herein to denote a solid inert surface or body to which an agent, such as a binding partner, that is reactive in any of the binding reactions described herein can be immobilized. The term “immobilized” as used herein denotes a molecularly-based coupling that is not dislodged or de-coupled under any of the conditions imposed during any of the steps of the assays described herein. Such immobilization can be achieved through a covalent bond, an ionic bond, an affinity-type bond, or any other covalent or non-covalent bond. Exemplary solid supports include chromatography resins and multi-well plates, or the substrate of the present disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DESCRIPTION OF CERTAIN EMBODIMENTS

The present disclosure relates to apparatuses and methods for detecting the amount and/or type of one or more analytes-of-interests such as biomarkers in a sample.

Further, the present invention relates to the fields of immunology and virology, including methods of detecting antibody formation and/or determining viral or vaccine induced immunity in a subject. In embodiments, the present disclosure relates to detecting a humoral response in a subject in response to an infectious agent or vaccine directed against an infectious agent. In embodiments, the present disclosure relates to apparatuses and methods for the quantitative detection of biomarkers against an infectious agent or vaccine, such as detecting one or more human antibodies against the SARS-CoV-2 virus, viral variants of SARS-CoV-2, and/or one or more vaccines directed against SARS-CoV-2, and variants of SARS-CoV-2.

In embodiments, the term SARS-CoV-2 includes virus from a reference strain of SARS-CoV-2 referred to as Wuhan-Hu-1 (GenBank accession MN908947) or ‘the original Wuhan strain’, sampled from a patient in Wuhan, China, on 26 Dec. 2019. As used herein the term SARS-CoV-2 includes variants of SARS-CoV-2. Although nomenclature for SARS-CoV-2 variants is not uniform one of ordinary skill in the art understands that established nomenclature for naming and tracking SARS-CoV-2 genetic lineages by GISAID, Nextstrain, and Pango are currently in use. Recently, the World Health Organization (WHO) recommended labeling SARS-CoV-2 variants using letters of the Greek Alphabet to refer to variants, such as variants of concern and variants of interest (See e.g., the world wide web at www.who.int/en/activities/tracking-SARS-CoV-2-variants/.). Accordingly, non-limiting examples of SARS-CoV-2 variants include variants of concern, or variants having an observed increase in transmissibility or detrimental change in the COVID 19 epidemiology, increase in virulence or change in clinical presentation, or decrease in effectiveness of preventative measures such as social distancing and vaccination. Non-limiting examples of current variants-of-concern include the Alpha, Beta, Gamma, and Delta (WHO labelled variants), or B.1.17 (documented first in the U.K.), B.1.351 (documented first in South Africa), P.1 (documented first in Brazil), B.1.617.2 (documented first in India) (Pango lineage), respectively. Variants of interest include SARS-CoV-2 isolate, that when compared to a reference isolate, its genome has mutations with established or suspected phenotypic implications. Non-limiting examples of current variants of interest include Epsilon, Zeta, Eta, Theta, Iota, and Kappa (WHO labelled variants), or B.1427/B1.429, P2, B.1.525, P3, B.1.526, B.1.6171.1 (Pango lineage), respectively. Variants also include natural or manmade variants of SARS-CoV-2 that have not yet formed, thus are not yet identified or named.

In some embodiments, SARS-CoV-2 refers to Wuhan-Hu-1 (GenBank accession MN908947), sampled from a patient in Wuhan, China, on 26 Dec. 2019 (See e.g., Wu F, Zhao S, Yu B, Chen Y-M, Wang W, Song Z-G et al. A new coronavirus associated with human respiratory disease in China. Nature. 2020; 579:265-9. doi: 10.1038/s41586-020-2008-3). That genome is 29 903 nucleotides (nt) in length and includes a gene order of similar structure to that seen in other coronaviruses: 5′-replicase ORF1ab-S-E-M-N-3′. The predicted replicase ORF1ab gene of Wuhan-Hu-1 is 21 291 nt in length. The ORF1ab polyprotein is predicted to be cleaved into 16 nonstructural proteins. ORF1ab is followed by a number of downstream open reading frames (ORFs). These include the predicted S (spike), ORF3a, E (envelope), M (membrane) and N (nucleocapsid) genes of lengths 3822, 828, 228, 669 and 1260 nt, respectively. Like SARS-CoV, Wuhan-Hu-1 also contains a predicted ORF8 gene (366 nt in length) located between the M and N genes. Finally, the 5′ and 3′ terminal sequences of Wuhan-Hu-1 are also typical of betacoronaviruses and have lengths of 265 nt and 229 nt, respectively. See e.g., Genomic sequencing of SARS-CoV-2: a guide to implementation for maximum impact on public health, 8 Jan. 2021, COVID-19: Laboratory and diagnosis available on the world wide web at www.who.int/publications/i/item/9789240018440.

In embodiments, the present disclosure relates to a method for determining a humoral response due to the presence of a target infectious agent or vaccine, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

In embodiments, the present disclosure relates to a method for determining a humoral response due to the presence of a target infectious agent or vaccine, including: capturing one or more biomarkers on a substrate that binds a plurality of biomarkers to a plurality of binding sites, wherein one or more, or at least two binding sites bind one or more or at least two biomarkers, and wherein when one or more biomarkers are present forming one or more bound biomarker(s)-of-interest; contacting the one or more bound biomarker(s)-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes. In embodiments, the process sequence further includes detecting emission light of the one or more fluorescent complexes. In embodiments, the process sequence may further determining a type and/or quantity of the one or more biomarkers or a plurality of biomarkers.

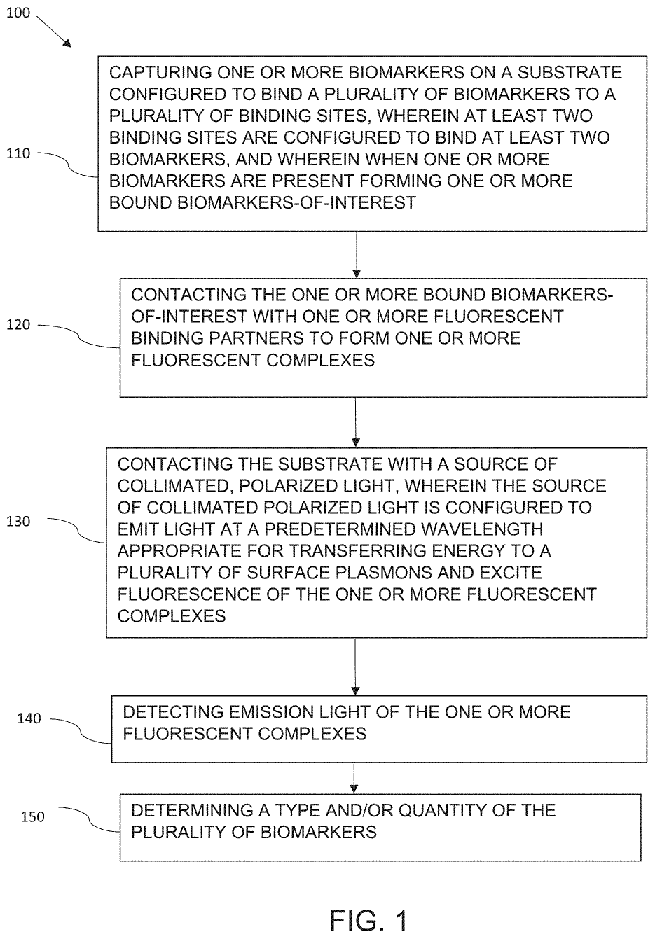

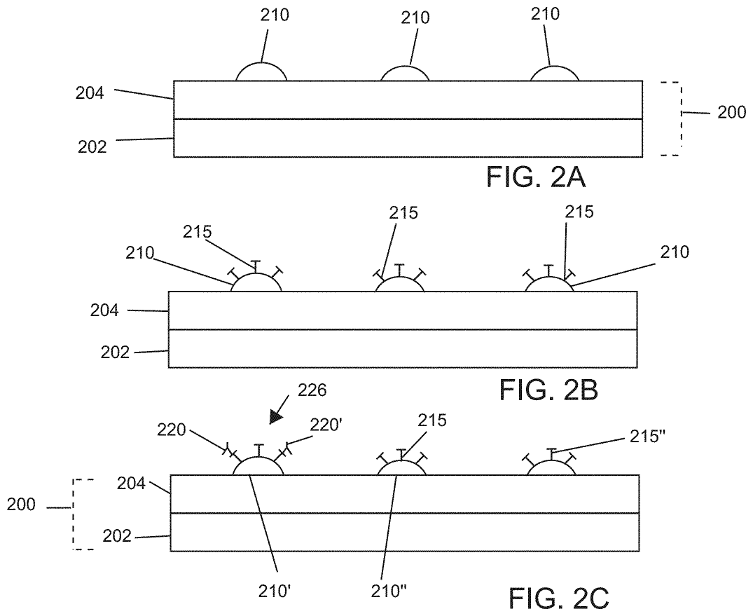

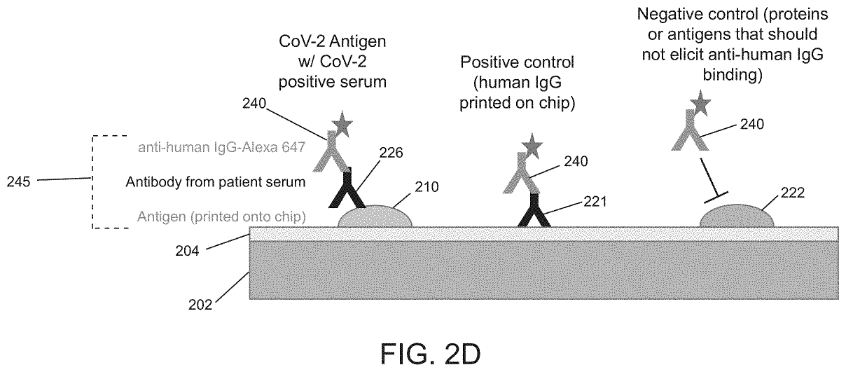

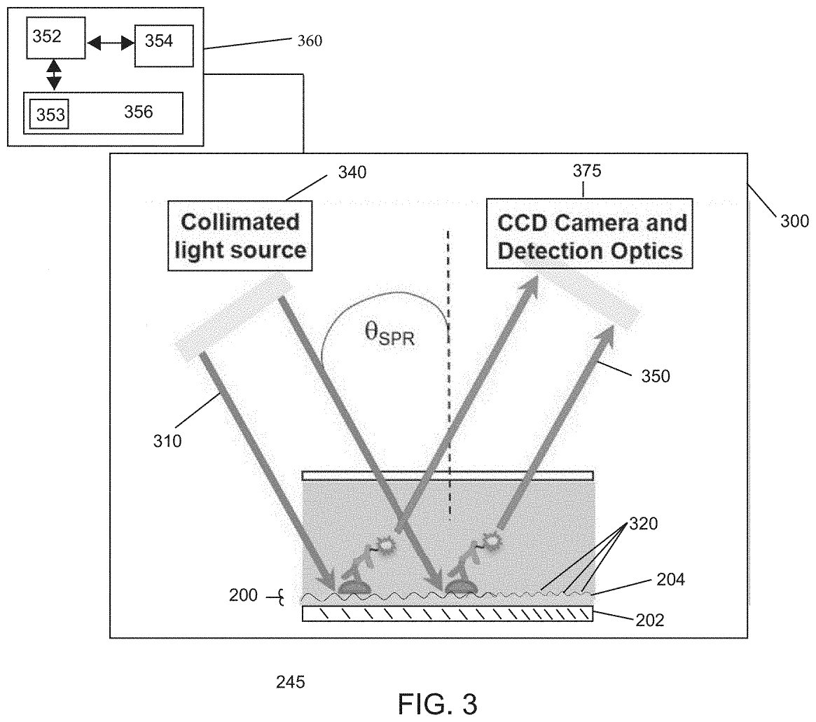

FIG. 1 is a flow diagram of a method 100 for a simultaneous qualitative and quantitative analysis of a plurality of biomarkers contained in a sample in accordance with some embodiments of the present disclosure. The method 100 is described below with respect to the stages of processing a substrate as depicted in FIGS. 2A-2D and may be performed, for example, in a suitable tool, such as grating-coupled fluorescent plasmonic (GC-FP) detection platform described below with respect to FIG. 3. Exemplary processing systems that may be used to perform the methods disclosed herein may include, but are not limited to, any of the GC-FP detection platforms, commercially available from Ciencia, Inc., of East Hartford, Conn. Other GC-FP detection platforms, including ones available from other manufacturers, may also be suitably used in connection with the teachings provided herein.

The method 100 is typically performed on a substrate 200 provided to a grating-coupled fluorescent plasmonic (GC-FP) detection platform described below with respect to FIG. 3. In some embodiments, as shown in FIG. 2A, the substrate 200 includes one or more layers such as first layer 202 and second layer 204 disposed atop the first layer 202. As shown in FIG. 2A, a plurality of, or one or more binding sites 210 are disposed atop the second layer 204. Although the following description is made with respect to three binding sites of the one or more binding sites 210 as shown, the substrate 200 may include any number of the one or more binding sites 210 configured to bind one or more (such as several) biomarkers as described below. In embodiments, binding sites may be configured as a positive or negative control depending upon experimental design needs, if any. In embodiments, the one or more binding sites 210 include one or more, or several antigens preselected to bind to a biomarker-of-interest.

In embodiments, the substrate 200 may include a first layer 202 including one or more of silicon (Si), silicon oxide (SiO₂), or the like. In embodiments, the first layer 202 is a polymer. In embodiments, the substrate 200 is not limited to any particular size or shape. The substrate 200 can be a round wafer having a 100 mm diameter, 150 mm diameter, 200 mm diameter, a 300 mm diameter or other diameters, such as 450 mm, among others. The substrate can also be any polygonal, square, rectangular, curved or otherwise non-circular workpiece. In some embodiments, as described further below, the substrate 200 is sized to fit within a test-tube.

In embodiments, substrate 200 includes a second layer 204 disposed atop the first layer 202. In embodiments, the second layer 204 is configured for use in a grating-coupled fluorescent plasmonic (GC-FP) detection platform and may include undulations of the first layer 202 and one or more metals such as a metal film including gold, silver or the like in a thickness sufficient to cover the first layer 202. Suitable thickness may include a thickness such as 1 to 100 nanometers, such as about 50 nanometers. In some embodiments, second layer 204 is a non-reactive metal such as gold layer disposed atop the first layer 202. In embodiments, as shown in FIG. 3, the second layer 204 may have a plurality of undulations 320 having a plurality of peaks and troughs in a predetermined depth and position. In embodiments, the substrate 200 includes additional layers of materials or may have one or more completed or partially completed one or more binding sites 210 such as a plurality of binding sites formed in or printed atop the substrate 200 such as directly atop the second layer 204 using any suitable deposition process known in the art. In some embodiments, the one or more binding sites 210 are disposed or printed directly atop second layer 204 and affixed atop second layer 204. In some embodiments, printing is performed as described in U.S. Pat. No. 9,400,353 to Cunningham et al. (herein entirely incorporated by reference). In some embodiments, the one or more binding sites 210 include one or more, several (3 to 6), or 3 or more (such as 3 to 20, 3 to 50, 3-100), or a plurality of binding partners such as antigens capable of undergoing a binding reaction with a biomarker to be analyzed such as a biomarker-of-interest. Biomolecule complex formation, antigen-antibody binding, or protein-protein binding is understood in the art. See e.g., U.S. patent publication nos. 2021/0139542 and 2019/0322739 (both of which are herein incorporated entirely by reference) for additional information relating to biomolecule binding interactions.

In embodiments, the one or more binding sites 210 are configured to bind a plurality of biomarkers to a plurality of binding sites, wherein at least two binding sites are configured to bind at least two biomarkers, and wherein when one or more biomarkers are present forming one or more bound biomarkers-of-interest. In embodiments, the one or more binding sites 210 bind or affix a plurality of biomarkers to a plurality of binding sites, wherein at least two binding sites are configured to bind at least two biomarkers, and wherein when one or more biomarkers are present forming one or more bound biomarkers-of-interest. For example, referring to FIGS. 2B and 2C, the one or more binding sites 210 may include one or more, several, or a plurality of antigens 215, 215″ or antibodies capable of undergoing an antigen-antibody reaction with one or more biomarkers to be analyzed such as a biomarker-of-interest contained in a subject's sample or specimen. As a result of the one or more binding sites 210 being fixed on the top surface of the second layer 204, the biomarkers-of-interest 220 are able to combine with the substrate 200 as shown in FIG. 2C. In embodiments, as shown in FIGS. 2C and 2D, fluorescent material 240 such as a dye, label or other predetermined fluorescent molecules may be applied to bind specifically to biomarker-of-interest 220.

In such embodiments, substrate 200 may be configured as a sensor film. In embodiments, substrate 200 is configured for high sensitivity to one or more biomarkers-of-interest 220, and the rate of the biomarker-binding partner reaction can be kept high. In some embodiments, the biomarker-of-interest 220 in a sample is to be detected by an antigen-antibody reaction, e.g., where an antigen (or an antibody) is fixed in or printed atop a metal film and an antibody (or an antigen) within the sample or specimen is to be detected, the antigen-antibody reaction may be promoted by preselecting the positioning of the one or more binding sites 210 atop second layer 204. In some embodiments, a plurality of biomarkers-of-interest such as 1 to 50, 1 to 30, 1 to 15, or 1 to 10 biomarkers-of-interest in a sample may be detected by antigen-antibody reactions, e.g., where a plurality of antigens (or antibodies) such as 1 to 50, 1 to 30, 1 to 15, or 1 to 10 antigens are fixed in a metal film and a plurality of biomarkers such as a plurality of antibodies (or antigens) such as 1 to 50, 1 to 30, 1 to 15, or 1 to 10 antibodies within the sample or specimen are to be detected, and multiple (such as 1 to 50, 1 to 30, 1 to 15, or 1 to 10) antigen-antibody reactions may be promoted atop one or more binding sites 210 atop second layer 204. As shown in FIG. 2D, a positive control binding partner 221 may also be applied or printed onto second layer 204, wherein the positive control binding partner 221 is configured to bind to fluorescent material 240 or binds fluorescent material 240. As shown in FIG. 2D, a negative control binding partner 222 may also be printed onto second layer 204, wherein the negative control binding partner 222 is configured not to bind to fluorescent material 240.

In some embodiments, substrate 200 is configured to bind a plurality of biomarkers to a plurality of the one or more binding sites 210, wherein one or more, or at least two binding sites (210′ and 210″ of FIG. 2C) of the one or more binding sites 210 are configured to bind, or bind at least two biomarkers when present, and wherein when one or more biomarkers are present forming one or more bound biomarkers-of-interest (See FIG. 2B, where biomarker 220 and biomarker 200′ are bound to binding site 210′). In embodiments, several or a plurality of bound-biomarkers-of-interest such as biomarker 220 and biomarker 220′ may be typed and quantified.

Non-limiting examples of suitable biomarkers according to the present disclosure include proteins-of-interest such as proteins for which expression is increased in a subject and have the potential to serve as informative indicators relating to disease or immunological status. In some embodiments, biomarkers refer to any measurable factor that differentiates a normal biological process from a disease related process or its response to therapy. In some embodiments, biomarkers may have a high diagnostic or prognostic performance. In some embodiments, biomarkers are proteins such as antibodies, cytokines, oligonucleotides specific to a target, or fragments thereof. Non-limiting examples of biomarkers include immunoglobulins such as one or more of immunoglobulin G (IgG), immunoglobulin M (IgM), immunoglobulin A (IgA), immunoglobulin (E), or isotypes or combinations thereof. In some embodiments, biomarkers include antibodies or fragments thereof selected from the group consisting of scFv, Fab, and a binding domain of an immunoglobulin molecule. In some embodiments, biomarkers provide diagnostic or prognostic indication of a specific disease. In some embodiments, biomarkers include biomarkers relating to Lyme Disease (LD) such as one or more antigens including: Borrelia burgdorferi outer surface protein (BBA69), plasminogen-binding protein (BBA70), laminin-binding protein (BmpA), Decorin binding proteins A and B (DbpA, DbpB), outer surface protein (ErpL), outer surface protein C (OspC), outer surface protein D (OspD), protein (P41), protein (P58), major protein (VMP)-like sequence (Vls) E lipoprotein (VIsE), or combinations thereof, see for example, Chou et al., A fluorescent plasmonic biochip assay for multiplex screening of diagnostic serum antibody targets in human Lyme disease, PLoS One. 2020; 15(2): e0228772 (herein entirely incorporated by reference). In embodiments, the biomarkers may be any biomarker. In embodiments, biomarkers include any biomarker known in the art such as those described in U.S. Pat. No. 8,026,049 (herein incorporated by reference).

In some embodiments, the one or more biomarkers include antibodies specific to SARS-CoV-2, fragments, or variants thereof. In some embodiments, biomarkers are characterized as serum antibody targets as known to one of ordinary skill in the art. In embodiments, antigens suitable for detection of COVID-19 related biomarkers using methods of the present disclosure include SARS-CoV-2 spike protein such as S1 (SEQ ID NO: 1) and fragments thereof such as the receptor binding domain (RBD); SARS-CoV-2 spike protein such as a protein characterized as S1S2 (SEQ ID NO: 2); or SARS-CoV-2 envelope protein such as N (SEQ ID NO: 3). In embodiments, biomarkers may include highly related sequences to these sequences such as polypeptides having at least 90%, 95%, 99% sequence identity to SEQ ID NO:1, SEQ ID NO: 2 or SEQ ID NO: 3. In embodiments, other proteins from SARS-CoV-2, and/or fragments or variants thereof may be antigens suitable for use in detecting SARS-CoV-2 related biomarkers-of-interest in accordance with the present disclosure. In embodiments, these antigens are disposed upon and bound to one or more binding sites such as binding site 210.

Referring back to FIG. 1, at process sequence 110 and FIG. 2C methods of the present disclosure include capturing one or more biomarkers-of-interest 220 on a substrate 200 configured to bind a plurality of biomarkers to a plurality of binding sites, wherein at least two binding sites such as binding site 210′ and 210″ are configured to bind at least two biomarkers, and wherein when one or more biomarkers are present forming one or more bound biomarkers-of-interest 226. In embodiments, a sample including one or more biomarkers-of-interest may be collected from a specimen, such as blood, blood serum, or dried blood. The sample may be contacted to substrate 200 under conditions in which one or more biomarkers-of-interest are able to contact and bind with one or more binding sites 210 as described here.

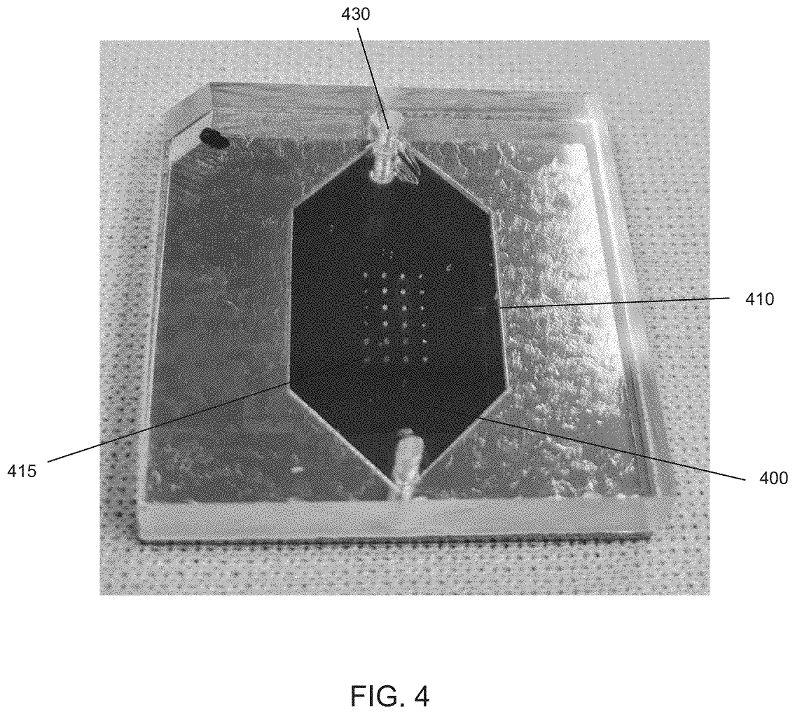

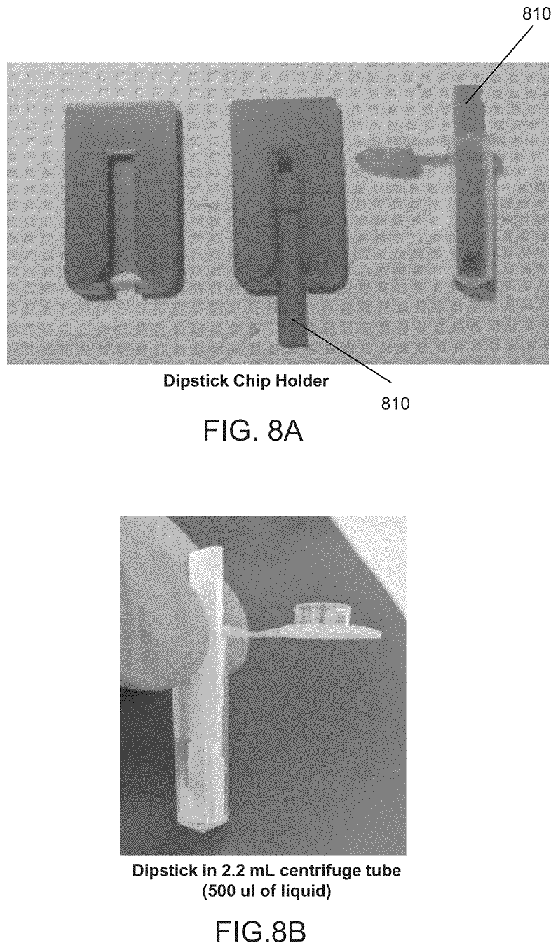

In some embodiments, the substrate 200 may be disposed within a cell such as a microfluidic flow cell, e.g., as shown in the photograph of FIG. 4 or a test-tube, e.g., as shown in FIG. 8B. In embodiments, a microfluidic flow cell such as GC-FP biochip 400 or test-tube is configured to accommodate a liquid such as a buffer solution. In embodiments, suitable buffer solution includes phosphate buffered saline supplemented with TWEEN-20 detergent (PBS-T).

Referring to FIG. 4, a photograph of a GC-FP biochip 400 is shown including a gasket 410 and a window 415 forming a microfluidic chamber, where serum samples and other reagents can be applied through an opening 430 and flowed over the second layer 204 (not shown in FIG. 4). In embodiments, a biomarkers-of-interest, when present, will be in fluid communication with the one or more binding sites 210. Upon contact, a bond may form such that the biomarker-of-interest is specifically fixed upon or captured by a corresponding binding partner such as in the antigen-antibody reaction described above.

Referring to FIG. 1, at process sequence 120 and FIG. 2D, methods of the present disclosure include contacting one or more bound biomarkers-of-interest 226 with one or more fluorescent binding partners 240 to form one or more fluorescent complexes 245. In embodiments, one or more fluorescent binding partners 240 are provided under conditions sufficient to bind to one or more bound biomarkers-of-interest 226. In embodiments, a GC-FP analysis is performed wherein a gold-coated biochip such as GC-FP biochip 400 is contacted with a fluorophore-labelled secondary antibody suitable for coupling with a surface plasmon field to emit enhanced fluorescent signal. Non-limiting examples of one or more fluorescent binding partners include fluorescent binding partners capable of emitting a fluorescent signal such as a dye including fluorescently labeled anti-human antibodies such as anti-human IgG-ALEXA FLUOR® 647, or other suitable antibody or dye such as those described in U.S. Patent Application No. 20080233660 (herein incorporated by reference). In embodiments, anti-human secondary antibodies suitable for use herein include affinity-purified antibodies with specificity for human immunoglobulins. In embodiments, a dye is preselected to be pH-insensitive over a wide molar range. In embodiments, the fluorescent material is in a labeling solution including buffer and a secondary antibody labeled with fluorescent material sufficient to provide a degree of labeling for each conjugate in the amount of 2-8 fluorophore molecules per antibody molecule. In embodiments, after the application of the labeling material, the top surface of the second layer 204 may be flushed or washed with buffer solution to remove any free, or non-bound fluorescent material.

Referring to FIG. 1, at process sequence 130 and FIG. 3 methods of the present disclosure include contacting the substrate 200 with a source of collimated, polarized light 310, wherein the source 340 of collimated polarized light 310 is configured to emit light at a predetermined wavelength appropriate for transferring energy to a plurality of surface plasmons and excite fluorescence of the one or more fluorescent complexes 226. For example, in embodiments, collimated, polarized light 310 is applied to the second layer 204 at a predetermined angle of incidence. In embodiments, the second layer 204 is configured as a periodic grating. In embodiments, the second layer 204 is a periodic grating or photonic grating. In embodiments, a suitable periodic grating or photonic grating for use herein facilitates or aides with momentum matching of the incoming polarized light (photons) to induce surface plasmon resonance. In embodiments, in the region irradiated with the light, the surface plasmon generated at second layer 204 couples with the fluorescence of the one or more fluorescent complexes 245 to emit enhanced fluorescent signal. In embodiments, when a fluorescent material is present in the region irradiated with the light, the fluorescence material is excited by the enhanced electric field formed by surface plasmon resonance, and fluorescence is emitted. In embodiments, the fluorescent signal is enhanced by greater than 10×, 10-100×, 100 to 5000×, such as 500 to 4000×, or 500 to 2000×.

In embodiments, a ROC analysis may be performed to determine a detection ratio value (mean signal/mean neg control+3 stdev) is indicative of a positive response. In embodiments, a value of 1 for detection ratio generally means that one has a positive response. However, in embodiments, due to the variability of the binding, one may need to go to a higher value to ensure 100% specificity, or alternatively, in other cases, one can go lower than 1 and still get 100% specificity.

Referring to FIG. 1, at process sequence 140 and FIG. 3 methods of the present disclosure include detecting emission light 350 of the one or more fluorescent complexes 245. In embodiments, the emitted fluorescent signal of emission light 350 is collected by a light detector such as a photodiode, a CCD image sensor, and detection optics 375. In embodiments, fluorescent enhancement can be achieved by preselecting a combination of wavelength and angle of incidence of the light 310.

Referring now to FIG. 1, at process sequence 150 methods of the present disclosure include determining a type and/or quantity of the plurality of biomarkers. In embodiments, the positioning of observed fluorescence may correspond to a predetermined type of biomarker. Thus, the binding sites are prepositioned on the substrate correlating to one or more specific biomarkers-of-interest. In embodiments, the fluorescence intensity as each binding site may correspond to the amount of detected biomarker such as detected antibody. In some embodiments, the amount of fluorescence intensity is determined by adjusting or normalizing image intensity such as that a positive control spot has an intensity of 100 (arbitrary units); finding the average fluorescence intensity of binding spots such as antigen spots (n=3 spots per chip); finding the average fluorescence intensity and standard deviation of one or more negative control spots (such as 2-3 spots of human serum albumin, aka: HSA); calculating a ratio of the (average binding spot intensity) vs. (negative control average spot intensity+3 standard deviations). In some embodiments, such as where testing for biomarkers-of-interest relating to COVID-19 such as antibodies specific to the polypeptides of SEQ ID NO:1, SEQ ID NO:2, or SEQ ID NO: 3, the calculating process sequence is repeated for each binding spot or antigen. In some embodiments, if 2 out of 3 of the ratios calculated are >1, then score=positive for antibodies against SARS CoV-2. In embodiments, if <2 out of 3 of the ratios are >1=negative for antibodies. In some embodiments, the stringency of the assay may be increased by requiring that at least 2 out of 3 are >1, and at least 1 ratio is >2.

In some embodiments, the present disclosure relates to a method for a qualitative and quantitative analysis of a plurality of biomarkers contained in a sample. In some embodiments, the quantitative and qualitative analysis are simultaneous. In some embodiments, the methods include capturing one or more biomarkers on a substrate configured to bind a plurality of biomarkers to a plurality of binding sites, wherein at least two binding sites bind, or are configured to bind, at least two biomarkers, and wherein when one or more biomarkers are present forming one or more bound biomarkers-of-interest. In some embodiments, the methods include contacting the one or more bound biomarkers-of-interest with one or more fluorescent binding partners to form one or more fluorescent complexes. In some embodiments, the methods include contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes. In some embodiments, the methods include detecting emission light of the one or more fluorescent complexes. In some embodiments, the methods include determining a type and/or quantity of the plurality of biomarkers. In some embodiments, the one or more biomarkers are immunoglobulins. In some embodiments, the immunoglobulins include one or more of immunoglobulin G (IgG), immunoglobulin M (IgM), immunoglobulin A (IgA), immunoglobulin E (IgE), or isotypes or combinations thereof. In some embodiments, the one or more biomarkers are serum antibody targets. In some embodiments, the one or more biomarkers are antigens comprising: BBA69, BBA70, BmpA, DbpA, DbpB, ErpL, OspC, OspD, P41, P58, VIsE, or combinations hereof. In some embodiments, the substrate is disposed within a cell. In some embodiments, the cell is a fluidic flow cell or a test-tube. In some embodiments, each binding site of the plurality of binding sites includes two or more predetermined antigens configured to bind to two or more different predetermined biomarkers-of-interest. In some embodiments, the plurality of biomarkers are derived from blood serum, blood plasma, whole blood, or dried blood. In some embodiments, the type is characterized as immunoglobulin G (IgG), immunoglobulin M (IgM), immunoglobulin A (IgA), or isotypes or combinations thereof. In some embodiments, the type is further characterized as virus specific to SARS-COV-2. In some embodiments, the plurality of biomarkers are antibodies specific to a SARS-COV-2 full-length spike protein (SEQ ID NO:1), SARS-COV-2 spike 1 protein (SEQ ID NO: 2, SARS-COV-2 nuclear envelope protein (N) (SEQ ID NO: 3). In some embodiments, contacting the substrate with a source of collimated, polarized light, further includes positioning the substrate within a grating-coupled fluorescent plasmonic (GC-FP) detection platform. In some embodiments, the substrate is further characterized as a GC-FP assay chip. In some embodiments, the source of collimated polarized light is configured to excite fluorescence of the one or more fluorescent complexes greater than 10×, 100×, greater than 500×, or greater than 1000×. In some embodiments, detecting excitation light of the one or more fluorescent complexes further includes forming a fluorescent image on an antigen array or map of antigens upon the substrate. In some embodiments, the substrate is disposed atop a dipstick configured to be deposited into a cell characterized as a tube. In some embodiments, the substrate is a 4×4 mm GC-FP chip.

Referring now to FIG. 3, device 300 may be a tool suitable to perform methods for processing a substrate in accordance with some embodiments of the present disclosure. In embodiments, systems that may be used to perform the methods disclosed herein may include, but are not limited to, any of the GC-FP detection platforms, described in U.S. Pat. No. 8,368,897 to Reilly et al. (herein entirely incorporated by reference). In some embodiments, an apparatus such as device 300 for qualitative and quantitative analysis of a plurality of biomarkers contained in a sample, includes: a substrate such as substrate 200 described above including a plurality of binding sites, wherein two or more of the plurality of binding sites include two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes. In some embodiments, device 300 is a grating-coupled fluorescent plasmonic (GC-FP) detection platform such as described in U.S. Pat. No. 8,368,897, wherein the platform is configured for contacting the substrate such as substrate 200 with a source of collimated, polarized light, wherein the source of collimated polarized light is configured to emit light at a predetermined wavelength appropriate for transferring energy to a plurality of surface plasmons and excite fluorescence of the one or more fluorescent complexes as described above.

In some embodiments, the substrate 200 is disposed within a cell. Referring now to FIGS. 8A and 8B, in some embodiments, the substrate is disposed atop a dipstick and configured to fit within the cell, and the cell is configured as a tube such as a test-tube. In some embodiments, the device 300 is a GC-FP detection platform including a detector such as such as a photodiode, a CCD image sensor, and detection optics 375 configured to detect emission light from one or more fluorescent complexes when one or more biomarkers-of-interest are contacted with a plurality of bonding sites. In some embodiments, substrate incudes substrate 200 including a plurality of binding sites, wherein each binding site of the plurality of binding cites includes two or more predetermined antigens configured to bind to two or more predetermined antibodies-of-interest. In some embodiments, the plurality of biomarkers are serum derived antibodies, antibodies from whole blood, or antibodies from dried blood.

Still referring to FIG. 3, to facilitate control of the device 300 as described above, a controller 360 may be provided as any form of general-purpose computer processor that can be used in an industrial setting for controlling various aspects of device 300. The memory, or computer-readable medium, 356 of a CPU 352 may be one or more of readily available memory such as random-access memory (RAM), read only memory (ROM), floppy disk, hard disk, or any other form of digital storage, local or remote. In some embodiments, support circuits 354 are coupled to a CPU 352 for supporting the processor in a conventional manner. In embodiments, these circuits may include one or more of cache, power supplies, clock circuits, input/output circuitry and subsystems, and the like. In some embodiments, the methods disclosed herein may generally be stored in the memory 356 as a software routine 358 that, when executed by the CPU 352, causes the device 300 to perform processes of the present disclosure. The software routine 358 may also be stored and/or executed by a second CPU (not shown) that is remotely located from the hardware being controlled by the CPU 352. Some or all of the method of the present disclosure may also be performed in hardware. As such, the disclosure may be implemented in software and executed using a computer system, in hardware as, e.g., an application specific integrated circuit or other type of hardware implementation, or as a combination of software and hardware. The software routine 358 may be executed after the substrate 200 is positioned in device 300. The software routine 358, when executed by the CPU 352, transforms the general-purpose computer into a specific purpose computer (controller 360) that controls the device 300 operation such that the methods disclosed herein are performed. In some embodiments, the present disclosure includes a computer readable medium having instructions stored thereon that, when executed, causes an apparatus for qualitative and quantitative analysis of a plurality of biomarkers contained in a sample to perform a method, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the computer readable media is non-transitory computer readable media.

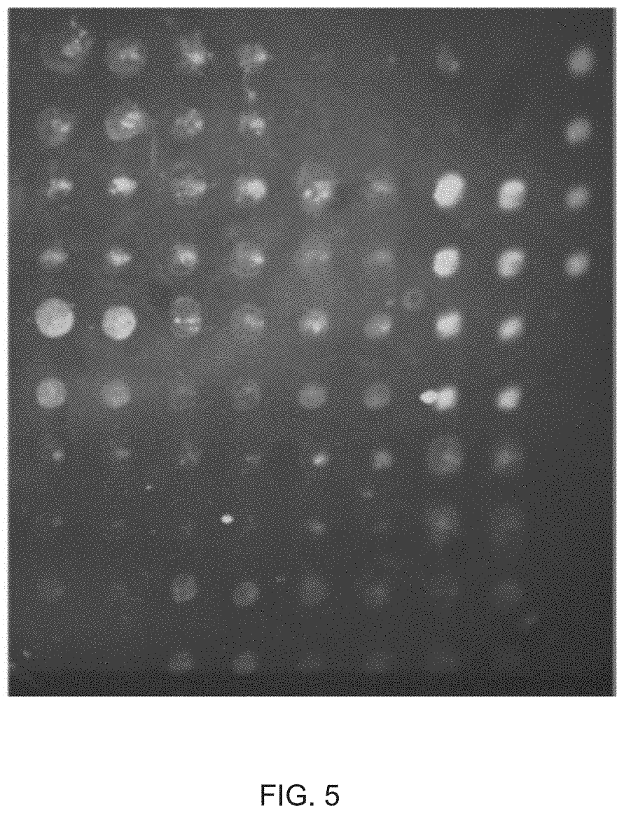

Referring now to FIG. 5, a photograph is shown of a GC-FP biochip image including a number of illuminated targets atop a plurality of binding sites. In embodiments, the fluorescence intensity at each binding site corresponds to the amount of detected antibody of various biomarkers-of-interest.

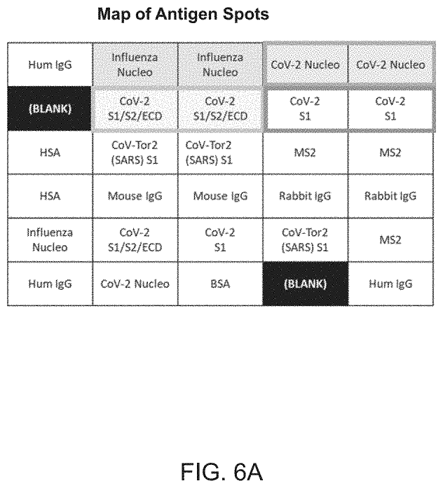

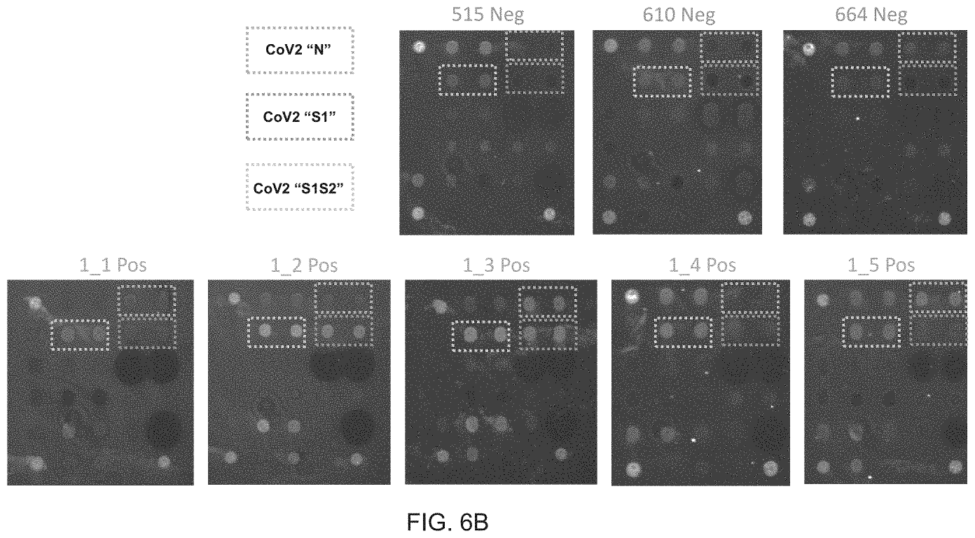

Referring now to FIGS. 6A and 6B, GC-FP chips processed with human blood serum are shown. FIG. 6A shows a map of antigen spots, and FIG. 6B shows images processed according to methods of the present disclosure. Three negative control samples (healthy individuals, with blood collected prior to COVID-19 pandemics were spotted onto binding sites at positions 515, 610, and 664. Five SARS-COV-2 positive samples were spotted (SARS-CoV-2 positive individuals confirmed with PCR test, all subjects were greater than 2 weeks convalescent). GC-FP enhanced fluorescent imaging of each chip for each sample was tested.

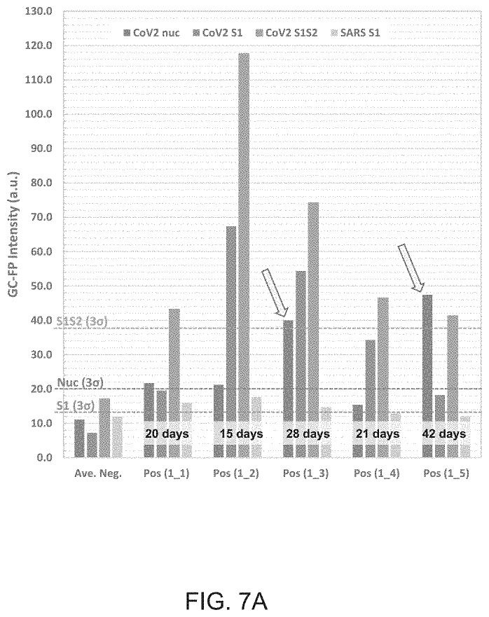

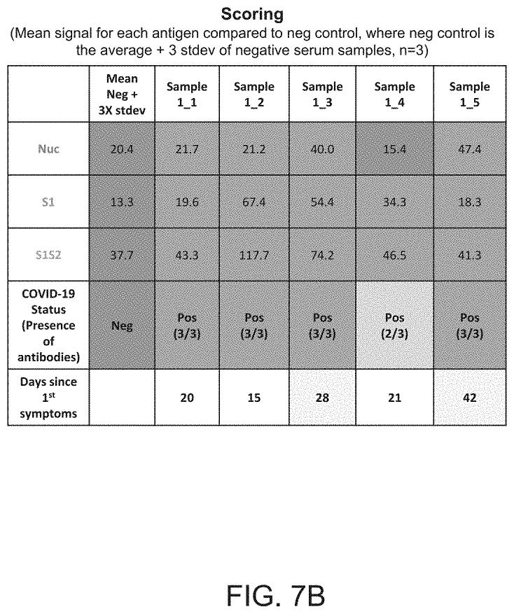

Referring now to FIGS. 7A and 7B, data from the images of FIGS. 6A and 6B was analyzed and scored. In embodiments, 3-sigma (Mean+3X std. deviation) for all three negative controls was determined for each target antigen (N, S1, and S1S2 of SARS-CoV-2). All positive samples were scored against these 3-sigma criteria. All positive samples showed greater than 3-sigma response for at least 2/3 of the target antigens (4 of 5 samples scored greater than 3-sigma for all 3 target antigens.

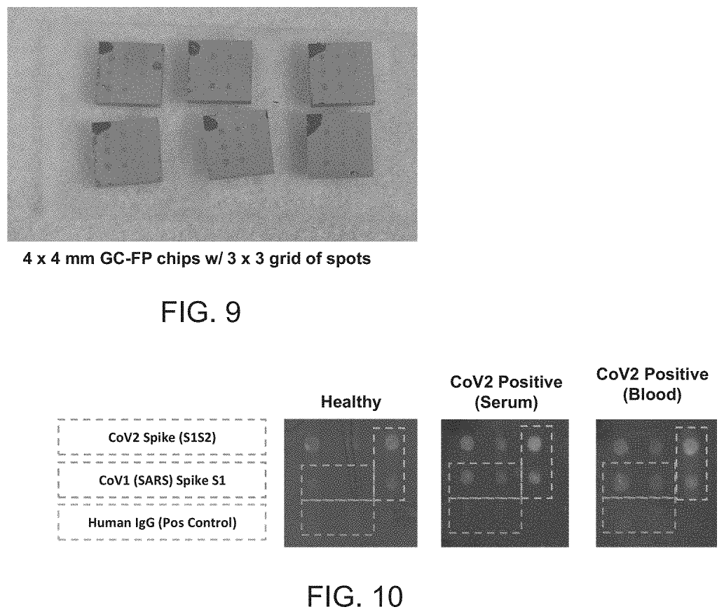

Referring now to FIGS. 8A and 8B a substrate 200 is shown disposed atop a dipstick 810. In embodiments, the dipstick is sized to fit within a 2.2 mL tube including at least 500 uL of liquid such as a buffer solution. Referring to FIG. 9 suitable substrate 200 are shown with predetermined binding spots suitable for antibody analysis in response to COVID-19. In embodiments, a 4×4 mm GC-FP chips are provided with 3×3 predetermined grid of binding sites or spots. In embodiments, the binding sites include one or more antigens for binding antibodies produced in a subject in response to SARS-CoV-2 infection. Referring to FIG. 10, images formed in accordance with the present disclosure are shown indicating negative or healthy, SARS-CoV-2 positive (from serum), and SARS-CoV-2 Positive from whole blood.

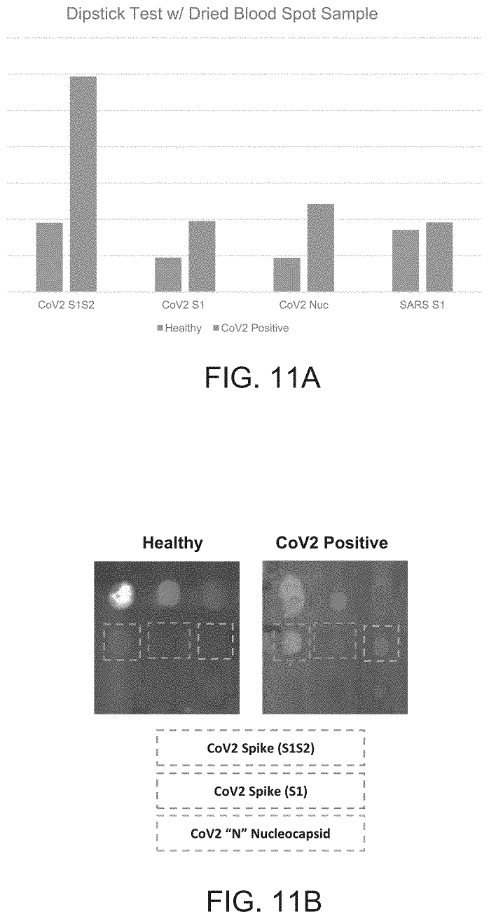

Referring now to FIGS. 11A and 11B, the dipstick format was applied to serum extracted from dried blood spots. In embodiments, dried blood spots were prepared on filter paper such as Whatman #4 paper (2 layers), Whatman 903 and stored longer than 24 hours at room temp or greater than 4 degrees Celsius. In embodiments, serum was extracted from 6 mm diameter dried blood spot samples (12 hrs. at 4 degrees Celsius) in a PBS-T buffer solution. In embodiments, the samples were applied to SARS-CoV-2 antibody test performed with dipstick GC-FP approach. Referring to FIGS. 11A and 11B, serum extracted from dried blood spots was a sufficient source for analyzing COVID-19 disease status. In embodiments, dried blood samples may be analyzed using a microfluidic chamber such as shown in FIG. 4.

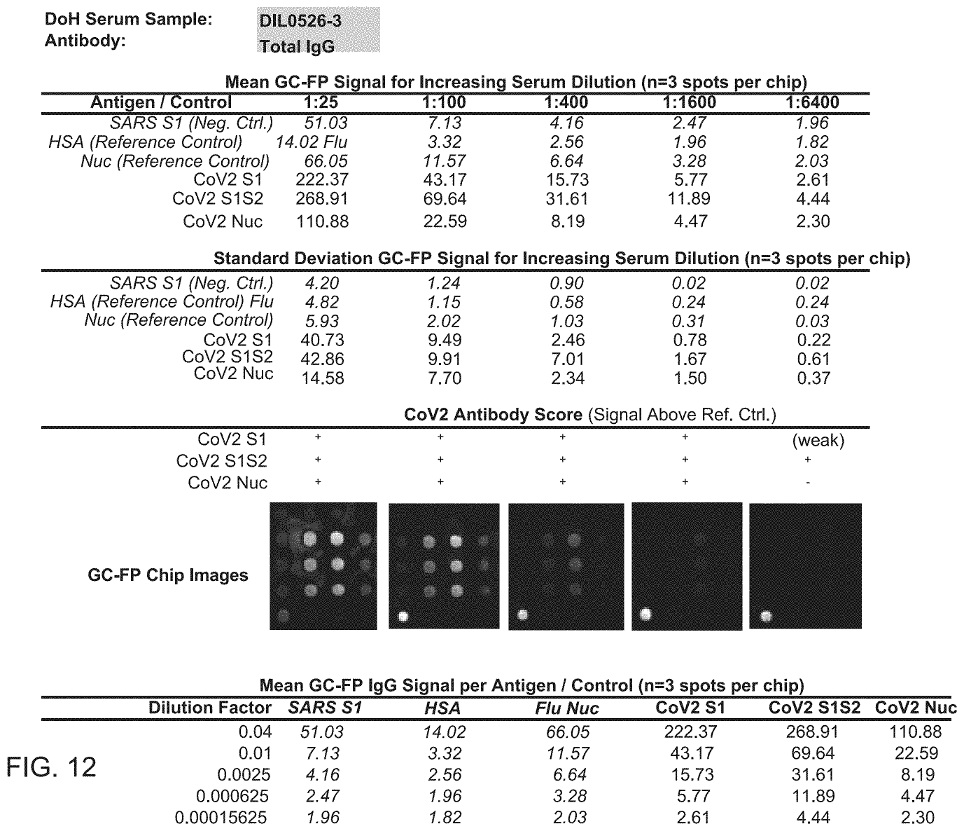

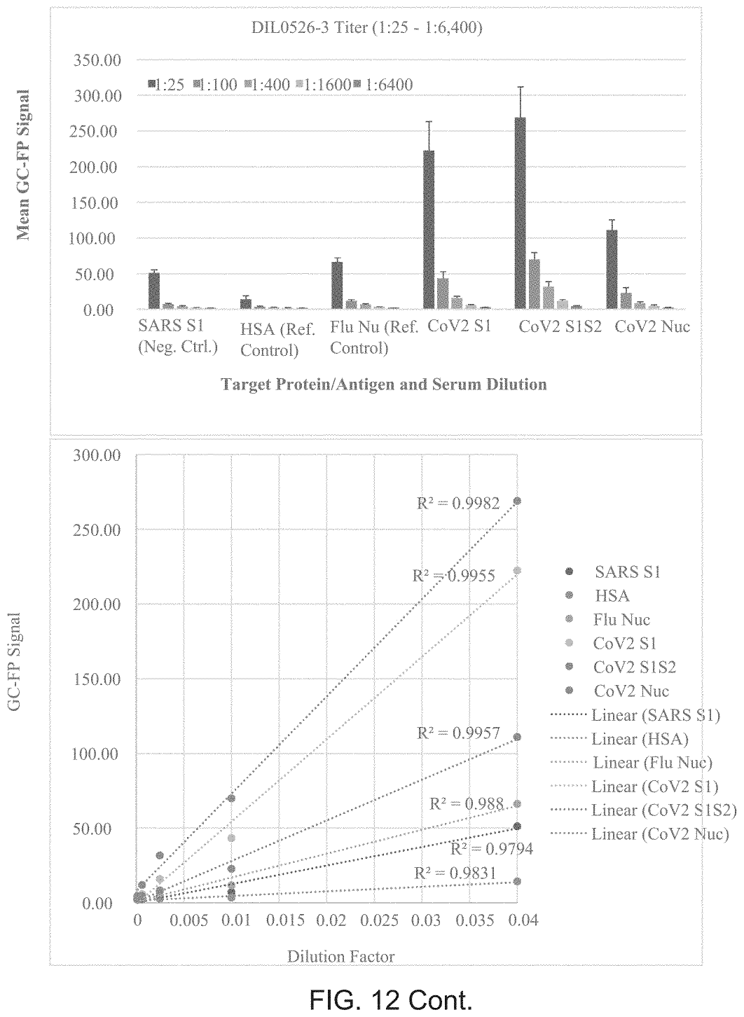

Referring to FIG. 12, GC-FP chips in accordance with the present disclosure were formed and provided robust indication of SARS-CoV-2.

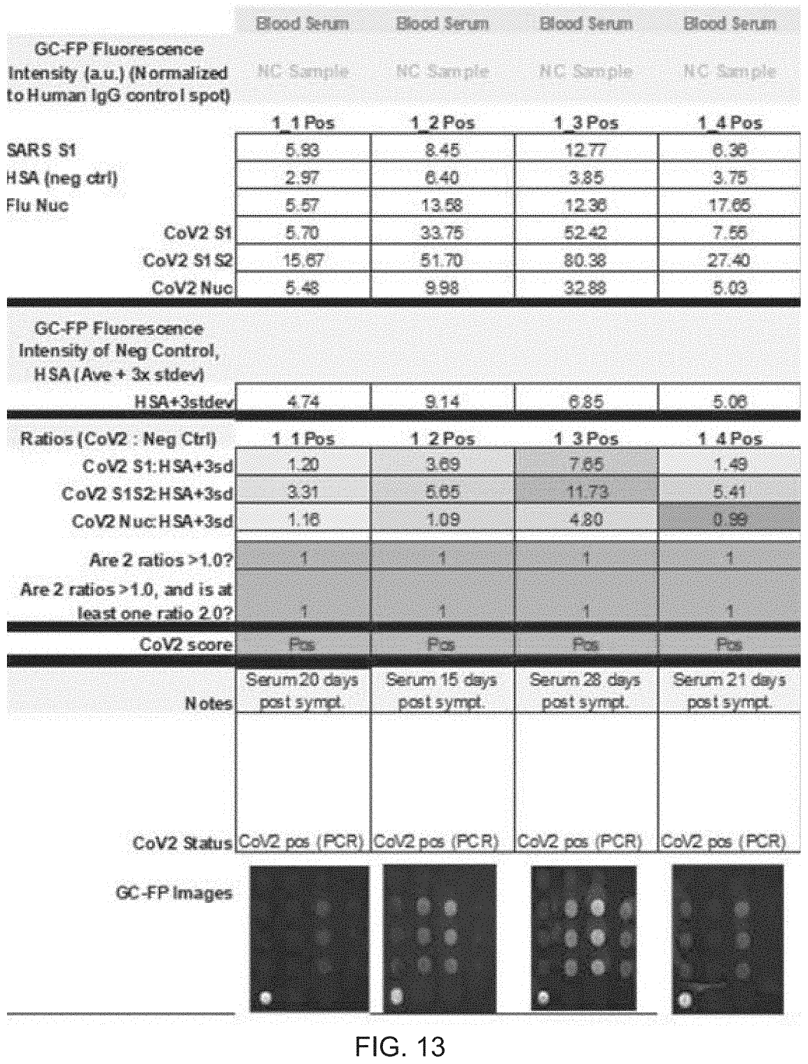

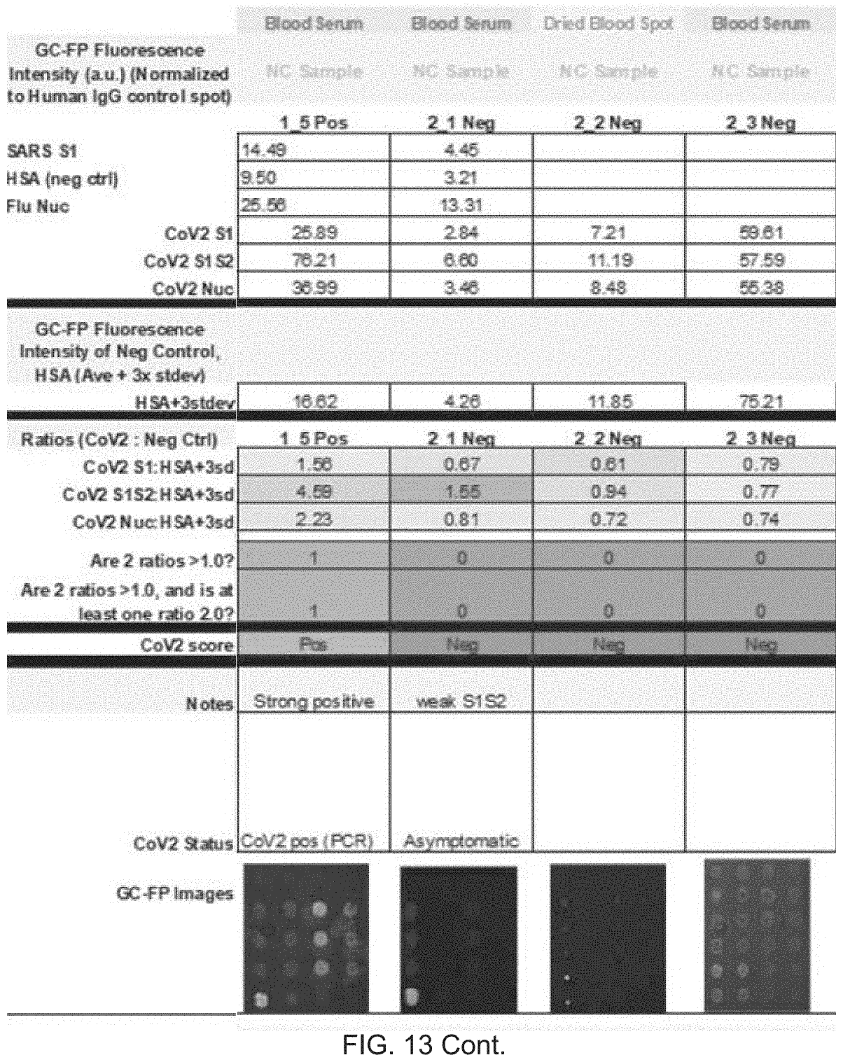

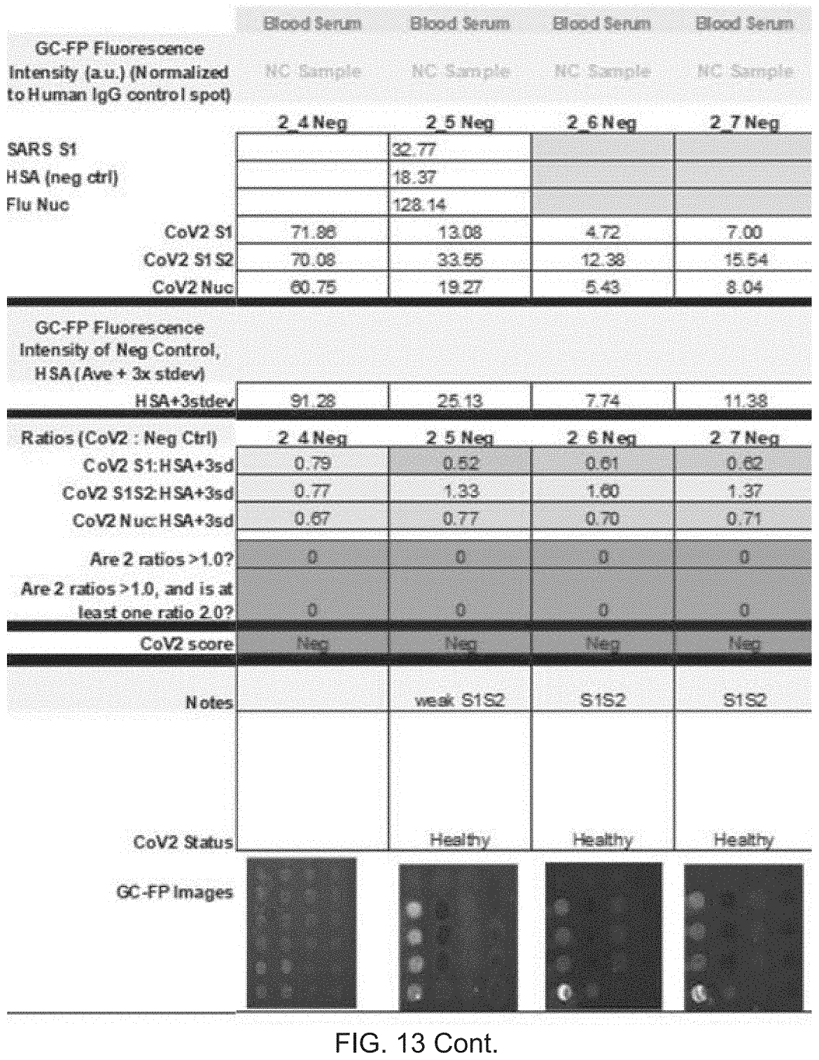

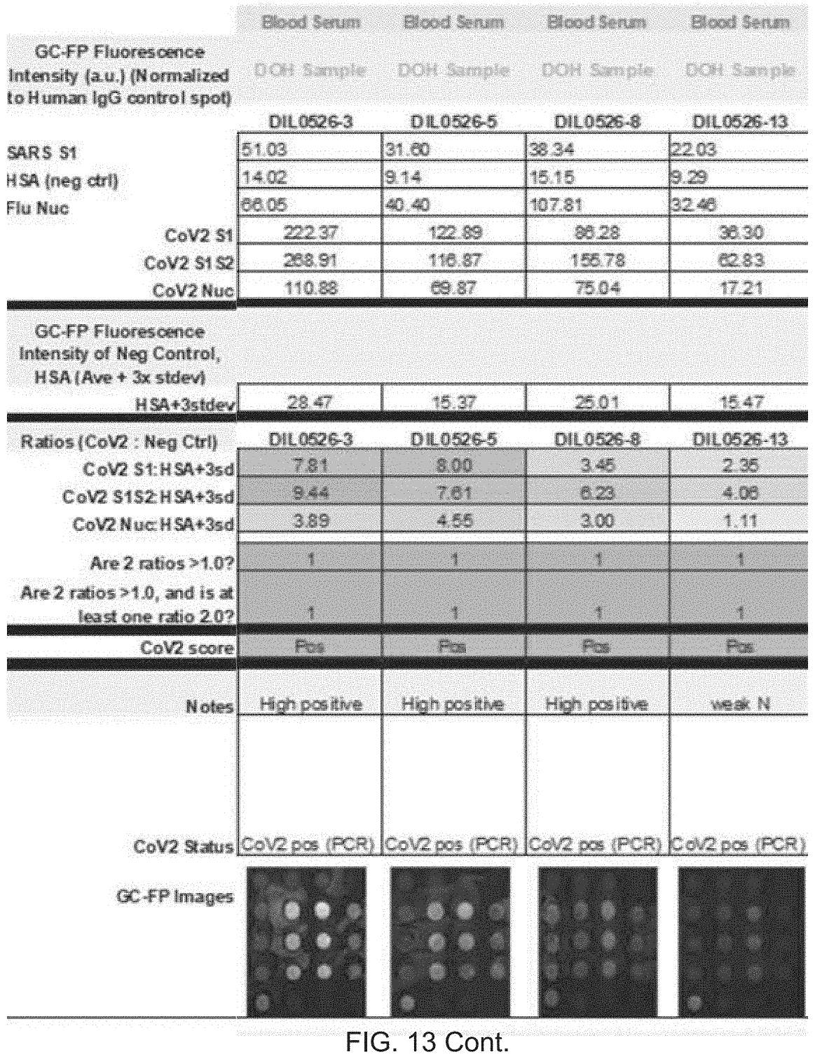

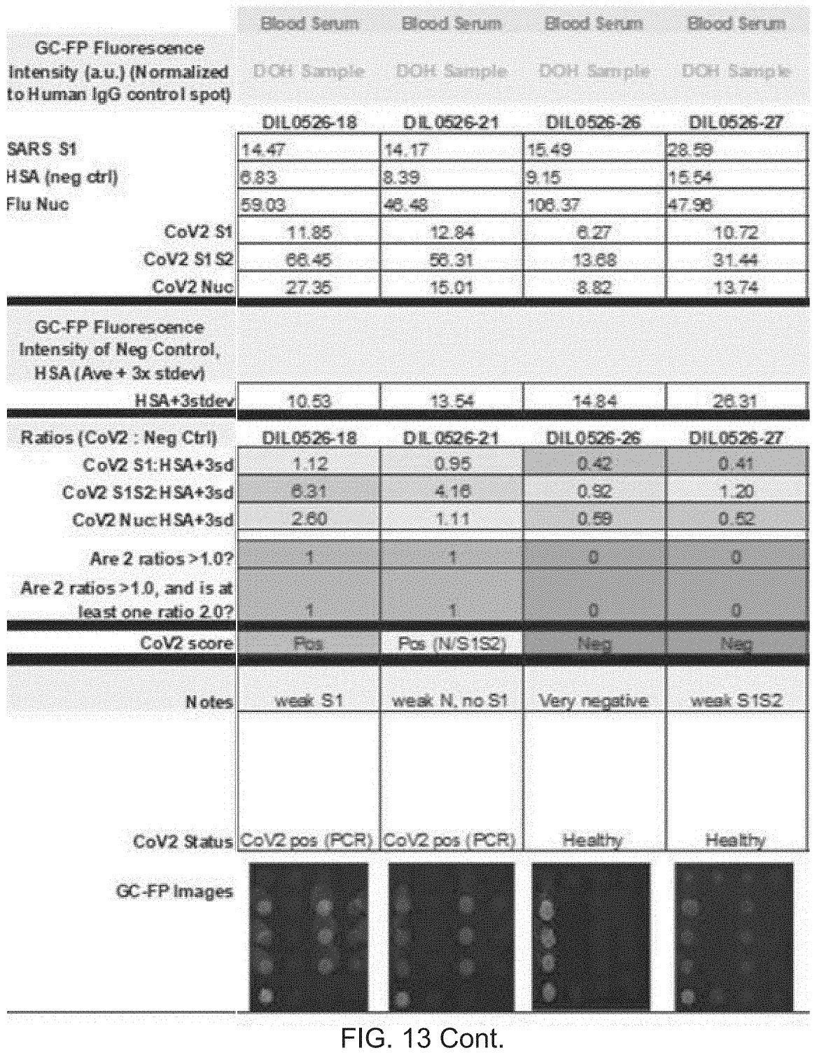

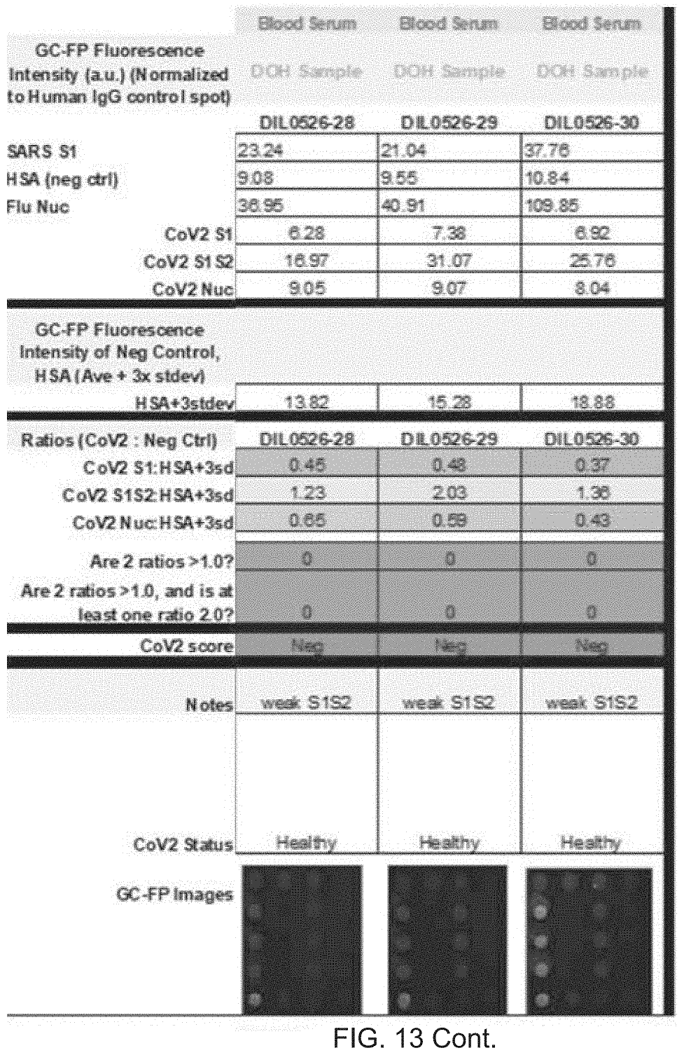

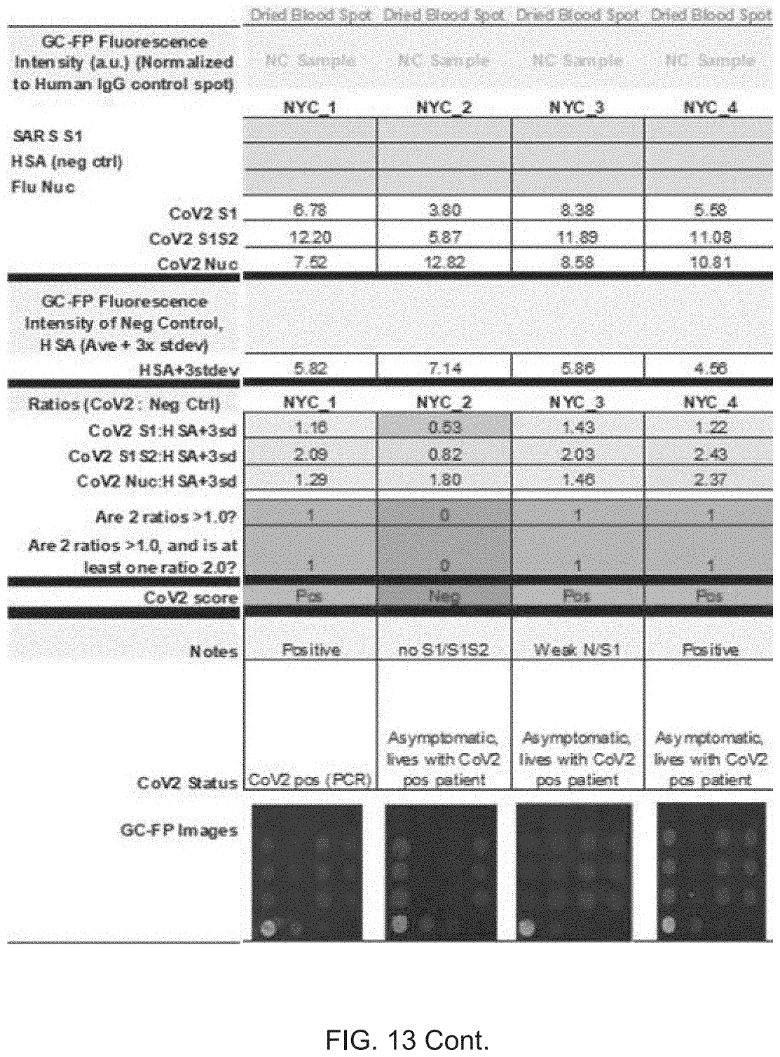

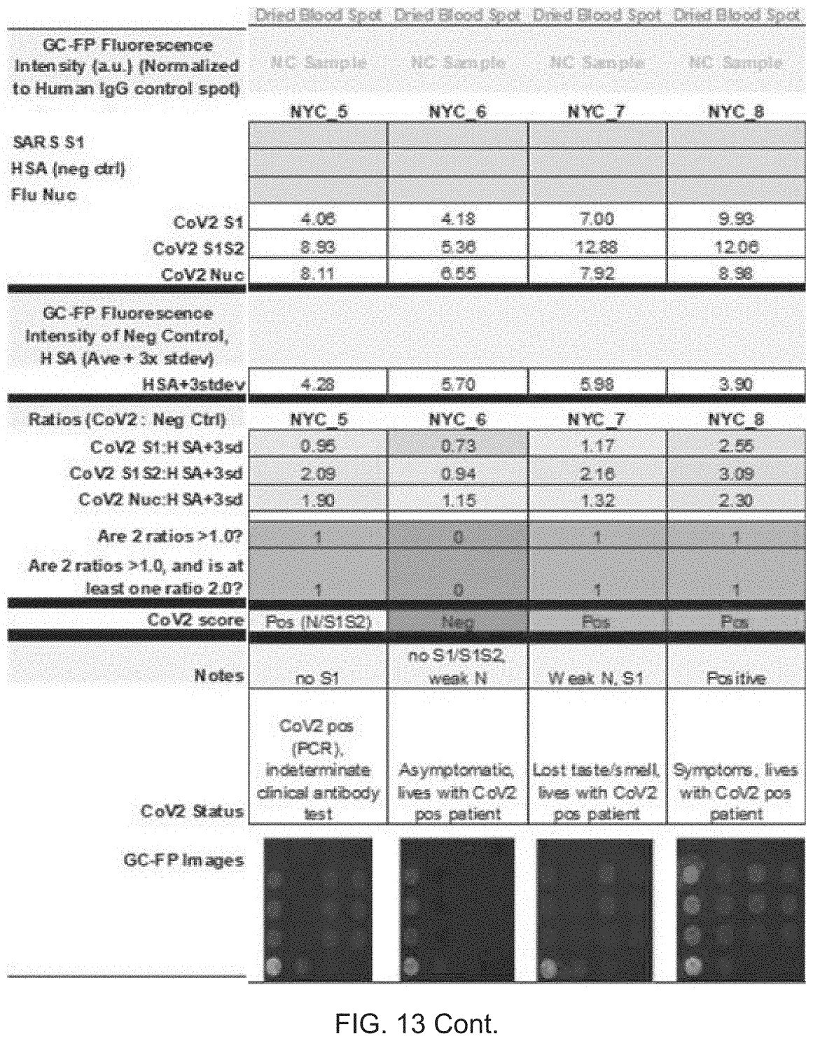

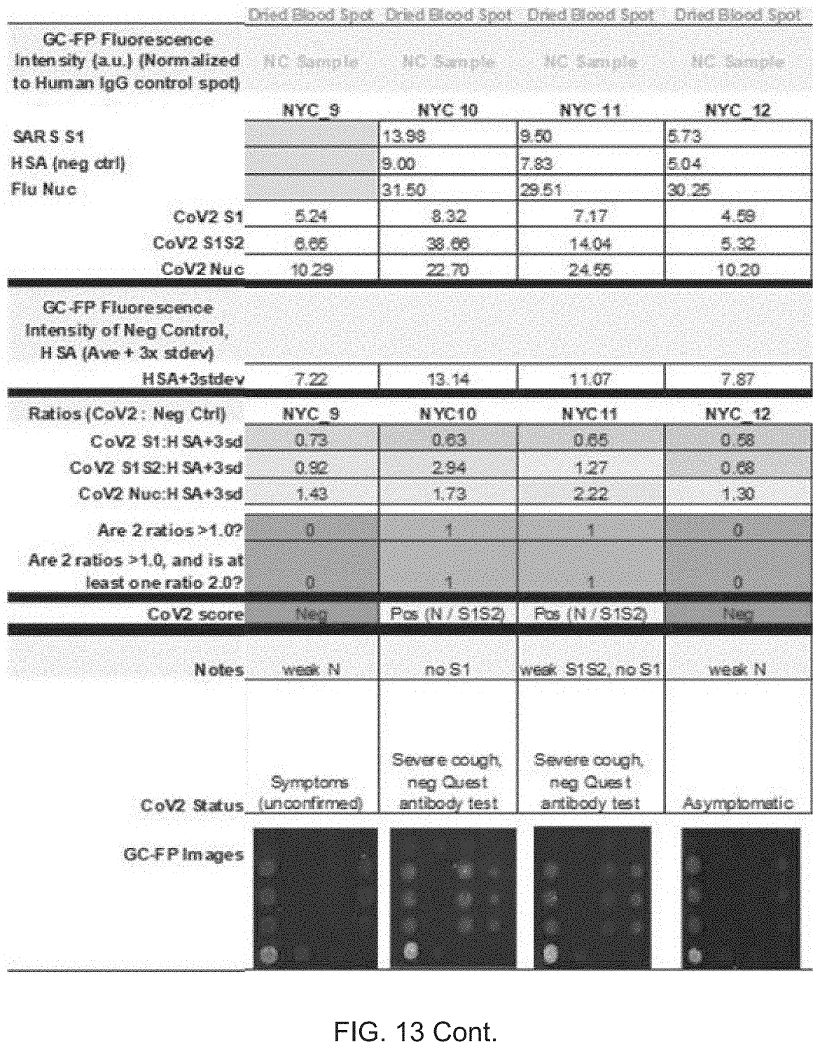

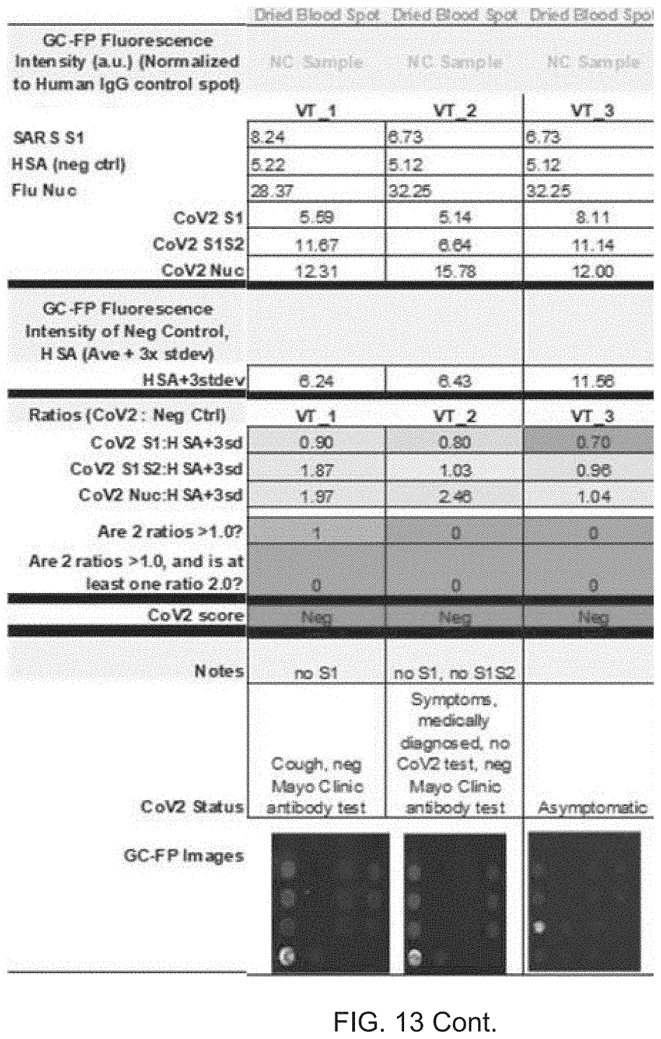

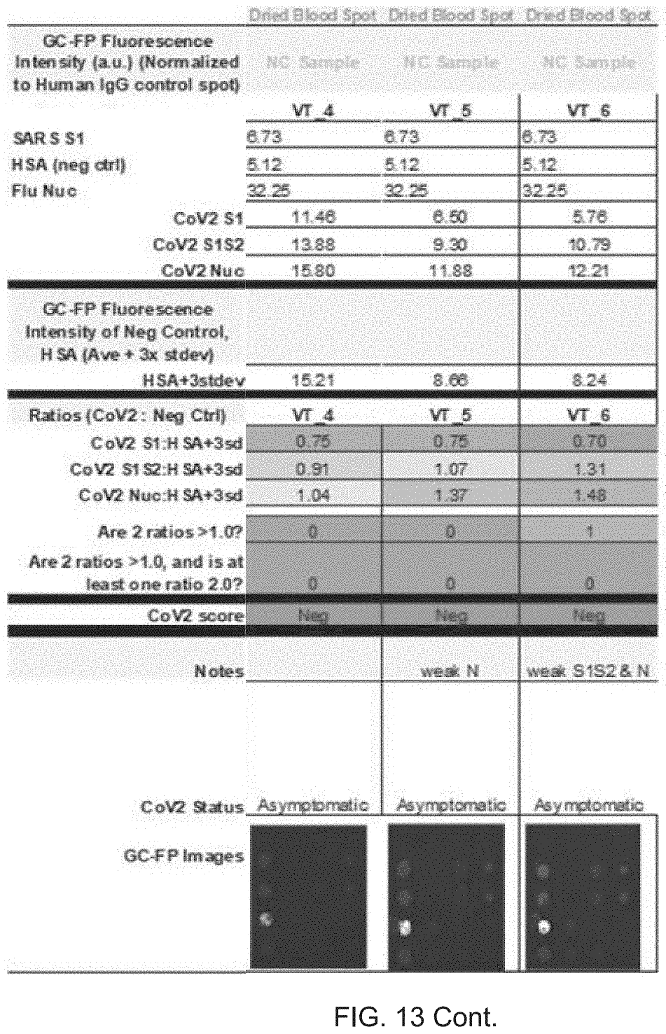

Referring to FIG. 13, GC-FP chips in accordance with the present disclosure were used to collect the sample data shown in FIG. 13.

In embodiments, the present disclosure relates to a method for a simultaneous qualitative and quantitative analysis of a plurality of biomarkers contained in a sample, including: capturing one or more biomarkers on a substrate configured to bind a plurality of biomarkers to a plurality of binding sites, wherein one or more, or at least two binding sites, bind or are configured to bind one or more, or at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the one or more biomarkers are immunoglobulins. In embodiments, the immunoglobulins comprise one or more of immunoglobulin G (IgG), immunoglobulin M (IgM), immunoglobulin A (IgA), or isotypes or combinations thereof. In embodiments, the one or more biomarkers are serum antibody targets. In embodiments, the one or more biomarkers are antigens including: BBA69, BBA70, BmpA, DbpA, DbpB, ErpL, OspC, OspD, P41, P58, VIsE, or combinations hereof. In embodiments, the substrate is disposed within a cell. In embodiments, the cell is a fluidic flow cell or a test-tube. In embodiments, each binding site of the plurality of binding sites includes two or more predetermined antigens configured to bind to two or more different predetermined biomarkers-of-interest. In embodiments, the plurality of biomarkers are derived from blood serum, blood plasma, whole blood, dried blood. In embodiments, the type is characterized as immunoglobulin G (IgG), immunoglobulin M (IgM), immunoglobulin A (IgA), or isotypes or combinations thereof. In embodiments, the type is further characterized as virus specific to SARS-COV-2. In embodiments, the plurality of biomarkers are antibodies specific to a SARS-COV-2 full-length spike protein (SEQ ID NO:1), SARS-COV-2 spike 1 protein (SEQ ID NO: 2, SARS-COV-2 envelope protein (N) (SEQ ID NO: 3). In embodiments, the substrate with a source of collimated, polarized light, further comprises positioning the substrate within a grating-coupled fluorescent plasmonic (GC-FP) detection platform. In embodiments, the substrate is further characterized as a GC-FP assay chip. In embodiments, the source of collimated polarized light is configured to cause fluorescent emission intensity greater than 10×, greater than 100×, greater than 500×, or greater than 1000×. In embodiments, detecting emission light of the one or more fluorescent complexes further comprises forming a fluorescent image on an antigen array or map of antigens upon the substrate. In embodiments, the substrate is disposed atop a dip-stick configured to be deposited into a cell characterized as a tube. In embodiments, the substrate is a 4×4 mm GC-FP chip.

In embodiments, the present disclosure relates to an apparatus for qualitative and quantitative analysis of a plurality of biomarkers contained in a sample, including: a substrate comprising a plurality of binding sites, wherein two or more of the plurality of binding sites comprises two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes; and a grating-coupled fluorescent plasmonic (GC-FP) detection platform, wherein the platform is configured for contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes. In embodiments, the substrate is disposed within a cell. In embodiments, the substrate is disposed atop a dip-stick and configured to fit within the cell, and wherein the cell is a test-tube. In embodiments, the GC-FP detection platform comprises a detector configured to detect emission light from one or more fluorescent complexes when one or more biomarkers-of-interest are contacted with a plurality of bonding sites. In embodiments, the substrate comprises a plurality of binding sites, wherein each binding site of the plurality of binding sites comprises two or more predetermined antigens configured to bind to two or more predetermined antibodies-of-interest. In embodiments, the plurality of biomarkers are serum derived antibodies, antibodies from whole blood, or antibodies from dried blood.

In embodiments, the present disclosure relates to a non-transitory computer readable medium having instructions stored thereon that, when executed, causes an apparatus for qualitative and quantitative analysis of a plurality of biomarkers contained in a sample to perform a method, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

In some embodiments, the present disclosure includes a method for determining a humoral response due to the presence of a target infectious agent or vaccine, comprising: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the one or more biomarkers are immunoglobulins, such as one or more of immunoglobulin G (IgG), immunoglobulin M (IgM), immunoglobulin A (IgA), or isotypes or combinations thereof. In some embodiments, the immunoglobulins comprise one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, the one or more biomarkers are serum antibody targets. In some embodiments, the substrate is disposed within a cell. In embodiments, the cell is a fluidic flow cell or a test-tube. In embodiments, each binding site of the plurality of binding sites comprises two or more predetermined antigens configured to bind to two or more different predetermined biomarkers-of-interest. In embodiments, the plurality of biomarkers are derived from blood serum, blood plasma, whole blood, dried blood. In embodiments, the type is characterized as immunoglobulin G (IgG), immunoglobulin M (IgM), immunoglobulin A (IgA), or isotypes or combinations thereof. In embodiments, the type is further characterized as virus specific to COVID-19, SARS-CoV-2, or a variant of SARS-CoV-2. In embodiments, the plurality of biomarkers are antibodies specific to a SARS-COV-2 full-length spike protein (SEQ ID NO:1), SARS-COV-2 spike 1 protein (SEQ ID NO: 2), SARS-COV-2 envelope protein (N) (SEQ ID NO: 3). In embodiments, contacting the substrate with a source of collimated, polarized light, further comprises positioning the substrate within a grating-coupled fluorescent plasmonic (GC-FP) detection platform. In embodiments, the substrate is further characterized as a GC-FP assay chip. In embodiments, the source of collimated polarized light is configured to cause fluorescent emission intensity greater than 10×, greater than 100×, greater than 500×, or greater than 1000×. In embodiments, detecting emission light of the one or more fluorescent complexes further comprises forming a fluorescent image on an antigen array or map of antigens upon the substrate. In embodiments, detecting emission light of the one or more fluorescent complexes further comprising normalizing a fluorescence intensity of a plurality of spots to a control spot. In embodiments, normalizing further comprises generating a GC-FP detection ratio. In embodiments, the substrate is disposed atop a dip-stick configured to be deposited into a cell characterized as a tube. In embodiments, the substrate is a 4×4 mm GC-FP chip.

In embodiments, the present disclosure includes an apparatus for quantitative analysis of a plurality of biomarkers contained in a sample, comprising: a substrate comprising a plurality of binding sites, wherein two or more of the plurality of binding sites comprises two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes; and a grating-coupled fluorescent plasmonic (GC-FP) detection platform, wherein the platform is configured for contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes, wherein the two- or more biomarkers-of-interest are selected from immunoglobulins comprising one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, the apparatus is configured to detect and normalize fluorescence of the one or more fluorescent complexes. In embodiments, the apparatus is configured to normalize fluorescence by generating a GC-FP detection ratio. In embodiments, the GC-FP detection platform comprises a detector configured to detect emission light from one or more fluorescent complexes when one or more biomarkers-of-interest are contacted with a plurality of bonding sites. In embodiments, the GC-FP detection platform comprises a detector configured to detect emission light from one or more fluorescent complexes when one or more biomarkers-of-interest are contacted with a plurality of bonding sites. In embodiments, the substrate comprises a plurality of binding sites, wherein each binding site of the plurality of binding sites comprises two or more predetermined antigens configured to bind to two or more predetermined antibodies-of-interest. In embodiments, the plurality of biomarkers are serum derived antibodies, antibodies from whole blood, or antibodies from dried blood.

In embodiments, the present disclosure includes a method for determining a humoral response due to the presence of a target infectious agent or vaccine, comprising: detecting in a biological sample of a subject at least one target antibody that is susceptible of being produced by the subject when the subject is vaccinated or infected by a target infectious agent, wherein the method includes: capturing the at least one target antibody on a substrate binds, or is configured to bind, the at least one target antibody to a plurality of binding sites, wherein at least two binding sites are configured to bind at least two target antibodies, and wherein when one or more target antibodies are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the at least one target antibody, wherein the at least one target antibody is selected from immunoglobulins comprising one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, determining the quantity of the at least one target antibody comprises generating a GC-FP diagnostic ratio. In embodiments, a grating coupled fluorescent plasmonic (GC-FP) assay was used to measure antibody levels that resulted from COVID-19 infection and/or vaccination. In embodiments, GC-FP can measure the level of antibodies resulting from vaccination, and that it can quantitatively measure the increase in antibody levels during the course of vaccination for multiple target antigens.

In some embodiments, the present disclosure includes a method of detecting antibodies to a human disease, comprising: vaccinating a subject with a vaccine to generate one or more antibodies to a predetermined infectious agent or variant thereof; capturing one or more antibodies on a substrate configured to bind the one or more antibodies to a plurality of binding sites, wherein at least two binding sites are configured to bind at least two antibodies, and wherein when one or more antibodies are present forming one or more bound antibodies-of-interest; contacting the one or more bound antibodies-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a quantity of the one or more antibodies. In embodiments, the one or more antibodies comprise at least one of immunoglobulin G (IgG), immunoglobulin M (IgM), immunoglobulin A (IgA), or isotypes or combinations thereof. In embodiments, the immunoglobulins comprise one or more of: immunoglobulin G (IgG) directed against SARS-CoV-2, a SARS-CoV-2 variant, and/or a COVID 19 vaccine; immunoglobulin M (IgM) directed against SARS-CoV-2, a SARS-CoV-2 variant, and/or a COVID 19 vaccine; immunoglobulin A (IgA) directed against SARS-CoV-2, a SARS-CoV-2 variant, and/or a COVID 19 vaccine, or isotypes or combinations thereof.

In embodiments, the present disclosure includes a method of measuring antibody levels that resulted from COVID-19 infection or vaccination against SARS-CoV-2 and variants thereof, comprising: (a) contacting a biological sample to a microchip-based grating coupled fluorescent plasmonic (GC-FP) assay; and (b) determining a quantity of antibodies directed against SARS-CoV-2, variants of SARS-CoV-2, or a vaccine directed against SARS-CoV-2. In embodiments, the methods further include vaccinating a subject in need thereof with a vaccine directed against SARS-CoV-2, or a variant thereof. In embodiments, the microchip-based grating coupled fluorescent plasmonic (GC-FP) assay comprises SARS-CoV-2 antigen, one or more antigens from one or more variant strains of SARS-CoV-2. In embodiments, the process sequence of (a) and (b) are repeated throughout a predetermined duration.

In embodiments, the present disclosure includes a method for determining a humoral response due to the presence of a target infectious agent or vaccine, including: capturing one or more biomarkers on a substrate configured to bind a plurality of biomarkers to a plurality of binding sites, wherein one or more binding sites bind one or more biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the biomarkers include immunoglobulins including one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.

In embodiments, the present disclosure includes a method for determining a humoral response due to the presence of a target infectious agent or vaccine, including: capturing one or more biomarkers on a substrate that binds a plurality of biomarkers to a plurality of binding sites, wherein one or more binding sites bind one or more biomarkers, and wherein when one or more biomarkers are present forming one or more bound biomarkers-of-interest; contacting the one or more bound biomarkers-of-interest with one or more fluorescent binding partners to form one or more fluorescent complexes. In embodiments, the process sequence includes contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes. In embodiments, the process sequence includes detecting emission light of the one or more fluorescent complexes; and determining a type and/or quantity of the plurality of biomarkers. In embodiments, the biomarkers include immunoglobulins including one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, the biomarkers include immunoglobulins including one or more of: immunoglobulin G (IgG) directed against SARS-CoV-2, or a SARS-CoV-2 variant; immunoglobulin M (IgM) directed against SARS-CoV-2, or a SARS-CoV-2 variant; immunoglobulin A (IgA) directed against SARS-CoV-2, or SARS-CoV-2 variant; or isotypes or combinations thereof.

In embodiments, the present disclosure includes a method for determining a humoral response due to a presence of a target infectious agent or vaccine. In embodiments, the methods includes sequentially, capturing one or more biomarkers on a substrate that binds a plurality of biomarkers to one or more binding sites, wherein one or more binding sites bind one or more biomarkers, and wherein when one or more biomarkers are present, forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the one or more biomarkers are immunoglobulins such as one or more of immunoglobulin G (IgG), immunoglobulin M (IgM), immunoglobulin A (IgA), or isotypes or combinations thereof. In embodiments, the immunoglobulins comprise one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, the one or more biomarkers are serum antibody targets. In embodiments, the substrate is disposed within a cell such as a fluidic flow cell or a test-tube. In embodiments, each binding site of the plurality of binding sites comprises two or more predetermined antigens that bind to two or more different predetermined biomarkers-of-interest. In embodiments, the plurality of biomarkers are derived from blood serum, blood plasma, whole blood, dried blood.

In embodiments, the present disclosure includes an apparatus for quantitative analysis of a plurality of biomarkers contained in a sample, including: a substrate including a plurality of binding sites, one or more of the plurality of binding sites includes one or more predetermined antigens that bind or are configured to bind to one or more predetermined biomarkers-of-interest and form one or more bound biomarkers-of-interest when biomarkers-of-interest are flowed over the substrate, and wherein the biomarkers-of-interest, when present, bind one or more fluorescent binding partners to form one or more fluorescent complexes; and a grating-coupled fluorescent plasmonic (GC-FP) detection platform, wherein the platform contacts the substrate with a source of collimated, polarized light, wherein the source of collimated polarized light emits light at a predetermined wavelength appropriate for transferring energy to a plurality of surface plasmons and excite fluorescence of the one or more fluorescent complexes, wherein the one or more biomarkers-of-interest are selected from immunoglobulins. In embodiments, the one or more of immunoglobulin G (IgG) are directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, the apparatus detects and normalizes fluorescence of the one or more fluorescent complexes. In embodiments, the apparatus is configured to normalize fluorescence by generating a GC-FP detection ratio. In embodiments, the GC-FP detection platform comprises a detector that detects, or is configured to detect emission light from one or more fluorescent complexes when one or more biomarkers-of-interest are contacted with a plurality of bonding sites. In embodiments, the plurality of biomarkers are serum derived antibodies, antibodies from whole blood, or antibodies from dried blood.

In embodiments, the present disclosure includes a method for determining a humoral response due to a presence of a target infectious agent or vaccine. In embodiments, the process includes sequentially: detecting in a biological sample of a subject at least one target antibody that is susceptible of being produced by the subject when the subject is vaccinated or infected by a target infectious agent, wherein the method comprises: capturing the at least one target antibody on a substrate that binds the at least one target antibody to a plurality of binding sites, wherein one or more binding sites bind one or more target antibodies, and wherein when one or more target antibodies are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light emits light at a predetermined wavelength appropriate for transferring energy to a plurality of surface plasmons and excite fluorescence of the one or more fluorescent complexes; detecting emission light of the one or more fluorescent complexes; and determining a type and/or quantity of the at least one target antibody, wherein the at least one target antibody is selected from immunoglobulins. In embodiments, the immunoglobulins include one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof. In embodiments, determining the quantity of the at least one target antibody includes generating a GC-FP diagnostic ratio. In embodiments, a grating coupled fluorescent plasmonic (GC-FP) assay was used to measure antibody levels that resulted from COVID-19 infection and/or vaccination. In embodiments, GC-FP can measure a level of antibodies resulting from vaccination, and it can quantitatively measure an increase in antibody levels during a course of vaccination for multiple target antigens

In embodiments, the present disclosure includes a method of detecting antibodies to a human disease. In embodiments, the method sequentially includes: vaccinating a subject with a vaccine to generate one or more antibodies to a predetermined infectious agent or variant thereof; capturing one or more antibodies on a substrate configured to bind the one or more antibodies to a plurality of binding sites, wherein one or more binding sites bind at least two antibodies, and wherein when one or more antibodies are present, forming one or more bound antibodies-of-interest; contacting the one or more bound antibodies-of-interest with one or more fluorescent binding partners to form one or more fluorescent complexes; contacting the substrate with a source of collimated, polarized light, wherein the source of collimated polarized light emits light at a predetermined wavelength appropriate for transferring energy to a plurality of surface plasmons and excite fluorescence of the one or more fluorescent complexes; detecting emission light of the one or more fluorescent complexes; and determining a quantity of the one or more antibodies. In embodiments, the one or more antibodies comprise at least one of immunoglobulin G (IgG), immunoglobulin M (IgM), immunoglobulin A (IgA), or isotypes or combinations thereof. In embodiments, the one or more antibodies comprise one or more of: immunoglobulin G (IgG) directed against SARS-CoV-2, a SARS-CoV-2 variant, and/or a COVID 19 vaccine; immunoglobulin M (IgM) directed against SARS-CoV-2, a SARS-CoV-2 variant, and/or a COVID 19 vaccine; immunoglobulin A (IgA) directed against SARS-CoV-2, a SARS-CoV-2 variant, and/or a COVID 19 vaccine, or isotypes or combinations thereof.

In embodiments, the present disclosure includes a method of measuring antibody levels that resulted from COVID-19 infection or vaccination against SARS-CoV-2 and variants thereof, including, sequentially: (1) contacting a biological sample to a microchip-based grating coupled fluorescent plasmonic (GC-FP) assay; and (2) determining a quantity of antibodies directed against SARS-CoV-2, variants of SARS-CoV-2, or a vaccine directed against SARS-CoV-2. In embodiments, vaccinating a subject in need thereof with a vaccine directed against SARS-CoV-2, or a variant thereof. In embodiments, steps (1) and (2) are repeated in sequence.

In embodiments, the present disclosure includes a method of detecting antibodies to a human disease, including: vaccinating a subject with a vaccine to generate one or more antibodies to a predetermined infectious agent or variant thereof; capturing one or more antibodies on a substrate configured to bind the one or more antibodies to a plurality of binding sites, wherein one or more binding sites bind at least two antibodies, and wherein when one or more antibodies are present, forming one or more bound antibodies-of-interest; contacting the one or more bound antibodies-of-interest with one or more fluorescent binding partners to form one or more fluorescent complexes; contacting the substrate with a source of collimated, polarized light, wherein the source of collimated polarized light emits light at a predetermined wavelength appropriate for transferring energy to a plurality of surface plasmons and excite fluorescence of the one or more fluorescent complexes; detecting emission light of the one or more fluorescent complexes; and determining a quantity of the one or more antibodies. In embodiments, the one or more antibodies comprise at least one of immunoglobulin G (IgG), immunoglobulin M (IgM), immunoglobulin A (IgA), or isotypes or combinations thereof. In embodiments, the one or more antibodies comprise one or more of: immunoglobulin G (IgG) directed against SARS-CoV-2, a SARS-CoV-2 variant, and/or a COVID 19 vaccine; immunoglobulin M (IgM) directed against SARS-CoV-2, a SARS-CoV-2 variant, and/or a COVID 19 vaccine; immunoglobulin A (IgA) directed against SARS-CoV-2, a SARS-CoV-2 variant, and/or a COVID 19 vaccine, or isotypes or combinations thereof.

In embodiments, the present disclosure includes an apparatus and method for measuring antibody levels that resulted from COVID-19 infection or vaccination against SARS-CoV-2 and variants thereof. In embodiments, the methods include (a) contacting a biological sample to a microchip-based grating coupled fluorescent plasmonic (GC-FP) assay; and (b) determining a quantity of antibodies directed against SARS-CoV-2, variants of SARS-CoV-2, or a vaccine directed against SARS-CoV-2. In embodiments, the methods further include vaccinating a subject in need thereof with a vaccine directed against SARS-CoV-2, or a variant thereof. In embodiments, the microchip-based grating coupled fluorescent plasmonic (GC-FP) assay includes SARS-CoV-2 antigen, one or more antigens from one or more variant strains of SARS-CoV-2. In embodiments, (a) and (b) are repeated throughout a predetermined duration. For example, (a) and (b) may be repeated 1-20 times over a period of weeks.

### Example I

Detecting Antibodies Directed Against the SARS-CoV-2 and Variants Thereof

Measuring the antibody response to 2019 SARS-CoV-2 is critical for diagnostic purposes, monitoring the prevalence of infection, and for gauging the efficacy of the worldwide vaccination effort COVID-19. Here, a microchip-based grating coupled fluorescent plasmonic (GC-FP) assay was used to measure antibody levels that resulted from COVID-19 infection and vaccination. In addition, the relative antibody binding towards antigens from variants CoV2 virus variants, strains B.1.1.7 (UK) and B.1.351 (S. African) were measured. Antibody levels against multiple antigens within the SARS-CoV-2 spike protein were significantly elevated for both vaccinated and infected individuals, while those against the nucleocapsid (N) protein were only elevated for infected individuals. GC-FP was effective for monitoring the IgG-based serological response to vaccination throughout the vaccination sequence, and could also resolve acute (within hours) increases in antibody levels. A significant decrease in antibody binding to antigens from the B.1.351 variant, but not B.1.1.7, was observed for all vaccinated subjects when measured by GC-FP as compared to the 2019 SARS-CoV-2 antigens. These results were corroborated by competitive ELISA assay. Collectively, the findings suggest that GC-FP is a viable, rapid, and accurate method for measuring both overall antibody levels to SARS-CoV-2 and relative antibody binding to viral variants during infection or vaccination.

The 2019 novel coronavirus, SARS-CoV-2 (COVID-19) has resulted in millions of deaths worldwide, and has spurred the development of novel diagnostic strategies, detection technologies, and vaccination approaches. Monitoring an individual's antibody responses to CoV2 antigens has become paramount from an epidemiological perspective, but also as a means of determining the efficacy of vaccination. By measuring the levels of antibodies (primarily IgG) in human blood or other bodily fluids, an individual's prior infection history, as well as their serological response to vaccination can be elucidated. Monitoring the stability and/or decline of antibody levels over time is important in estimating how long individuals will retain immunity (See e.g., Chia, W. N., Zhu, F., Ong, S. W. X., et al., 2021. Lancet Microbe 2(6), E240-E249). Beyond assessing human serological response to infection or vaccination, it is important to understand how an individual's immune system will respond to a growing number of novel SARS-CoV-2 variants. A core area of concern is mutations in the spike protein, which could interfere with antibody binding, and subsequently affect the blockade (neutralization) of viral entry into human cells via the ACE2 receptor (See e.g., Hoffmann, M., Arora, P., Groß, R., et al., 2021. Cell 184(9), 2384-2393.e2312.) This could ultimately result in breakthrough infections for individuals who were previously infected or vaccinated (Hoffmann et al., 2021). Recent studies have shown that emerging variants in the United Kingdom (B.1.1.7) and South Africa (B.1.351) are not as effectively neutralized by blood serum from vaccinated individuals, nor from those who were previously infected with the original 2019 SARS-CoV-2strain (See e.g., Chang, X., Augusto, G. S., Liu, X., et al., 2021. bioRxiv, 2021.2003.2013.435222; Hoffmann, M., Arora, P., Groβ, R., et al., 2021. Cell 184(9), 2384-2393.e2312; and Xie, X., Liu, Y., Liu, J., et al., 2021. Nat Med 27(4), 620-621). This is also an area of concern for variants emerging in other regions, including Brazil and India (See e.g., Mahase, E., 2021. BMJ 372, n158, and Wise, J., 2021. BMJ 373, n1315). This highlights the need for sensitive, specific, high-throughput assays to monitor antibody levels and predict effectiveness.

Multiplexed quantitative serological assays; allow both the determination of antibody levels in response to infection and vaccination, and the ability to assess relative antibody neutralizing capacity, provide an attractive diagnostic solution. Established methods of assessing antibody response to SARS-CoV-2 and its variants include cell-based viral neutralization assays (Hoffmann, M., Arora, P., Groß, R., et al., 2021. Cell 184(9), 2384-2393.e2312; Muruato, A. E., Fontes-Garfias, C. R., Ren, P., et al., 2020. Nat Commun 11(1), 4059; Tan, C. W., Chia, W. N., Qin, X., et al., 2020. Nat Biotechnol 38(9), 1073-1078; and Xie, X., Liu, Y., Liu, J., et al., 2021. Nat Med 27(4), 620-621) and competitive in vitro binding assays such as ELISA (See e.g., Peterhoff, D., Glück, V., Vogel, M., et al., 2021. Infection 49(1), 75-82, and Valdivia, A., Torres, I., Latorre, V., et al., 2021. Eur J Clin Microbiol Infect Dis 40(3), 485-494). Previously, a grating-coupled fluorescent plasmonic (GC-FP) biosensor platform for rapid (30 min), quantitative, multiplexed detection of human antibody response to both Lyme disease and COVID-19 infection (See e.g., Cady et al., 2021; and Chou, E., Lasek-Nesselquist, E., Taubner, B., et al., 2020. PLoS One 15(2), e0228772) has been demonstrated. The GC-FP detection ratio (ratio of antibody binding to target antigens vs. negative control proteins) for human serum and dried blood spot samples correlated strongly with standard antibody testing approaches, including microsphere immunoassay (MIA) and ELISA (Cady et al., 2021). Notably, it was found that dried blood spot samples as well as more traditional blood serum samples could yield high sensitivity and selectivity for diagnosing prior COVID-19 infection.

Here, GC-FP detection microchips are modified to include additional antigens from variant strains of SARS-CoV-2, including B.1.1.7 and B.1.351. Using these new detection microchips, it is now possible to simultaneously monitor antibody levels against multiple 2019 SARS-CoV-2 antigens for individuals throughout the vaccination sequence, and for all three vaccine types currently approved for use in the United States: Pfizer-BioNTech (See e.g., Polack, F. P., Thomas, S. J., Kitchin, N., et al., 2020. N Engl J Med 383(27), 2603-2615), Moderna (See e.g., Baden, L. R., El Sahly, H. M., Essink, B., et al., 2021. N Engl J Med 384(5), 403-416) and Johnson & Johnson (See e.g., Sadoff, J., Gray, G., Vandebosch, A., et al., 2021. N Engl J Med 384, 2187-2201). The relative binding of antibodies to original and variant strains of SARS-CoV-2 antigens for multiple exposure scenarios including: 1) acute severe (hospitalized) infection, 2) mild infections that did not lead to hospitalization, 3) vaccination with all three vaccines, 4) and a combination of prior infection and vaccination have also been assessed. The results demonstrate that GC-FP is an effective and sensitive method to monitor antibody levels in response to vaccination, and can determine relative antibody binding levels to original and variant 2019 SARS-CoV-2 antigens, all in a single, rapid test.

### Materials and Methods

### Materials

Nucleocapsid protein (N), the S1 fragment of the spike protein (S1), the extracellular domain of the spike protein (S1S2), the receptor binding domain of the spike protein (RBD) for the 2019 SARS-CoV-2 virus, S1 variant antigens, RBD variant antigens, and human serum albumin (HSA) were all obtained from Sino Biological, Inc. (Table 1). Positive control protein, human IgG protein (Hum IgG), SuperBlock blocking buffer and phosphate buffered saline (PBS) were obtained from ThermoFisher Scientific. PBS-TWEEN (PBS-T) solution consisting of PBS+0.05% v/v TWEEN-20 (Sigma-Aldrich) was prepared on a daily basis for all experiments. Alexa Fluor 647 labeled anti-human IgG (heavy and light chain) were obtained from Invitrogen/ThermoFisher Scientific. ACE2 competitive ELISA testing was performed using COVID-19 ACE2 testing kits from RayBiotech (COVID-19 Spike Variant-ACE2 Binding Assay Kit).

### Grating-Coupled Fluorescent Plasmonic (GC-FP) Biosensor Chip Preparation

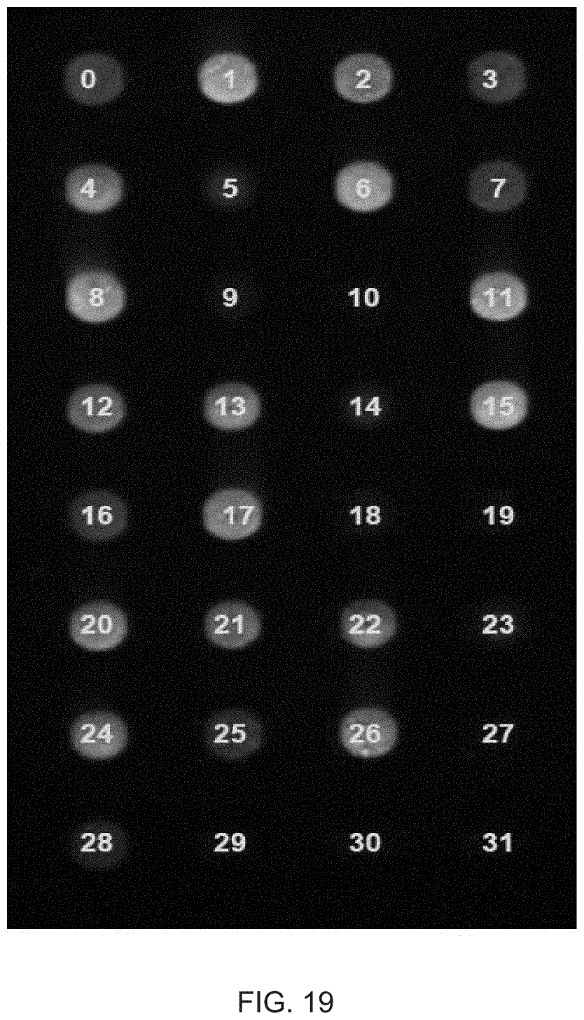

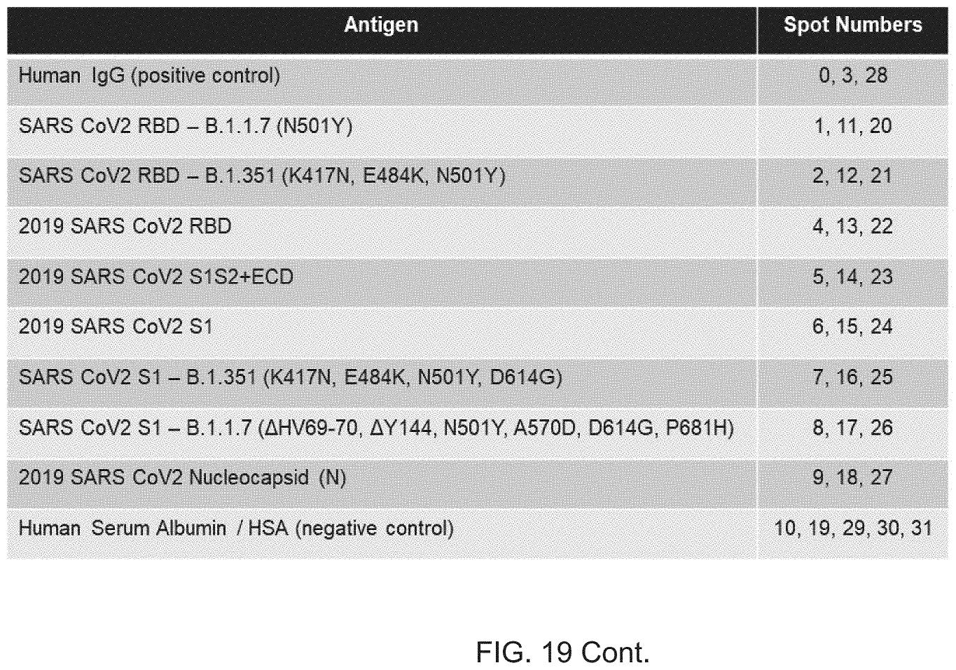

Gold coated grating-coupled fluorescent plasmonic (GC-FP) biosensor chips were fabricated as described our previous work (Cady et al., 2021; Chou, E., Lasek-Nesselquist, E., Taubner, B., et al., 2020. PLoS One 15(2), e0228772, and Chou, E., Pilar, A., Guignon, E., et al., 2019. SPIE BiOS 10895). GC-FP chips were also printed as described previously (Cady, N.C., Tokranova, N., Minor, A., et al., 2021. Biosens Bioelectron 171, 112679). A map of the protein/antigen spots is shown in FIG. 19.

TABLE 1
Proteins and peptides used for generating
GC-FP detection microchips.

| Antigen/Protein | Source |
|---|---|
| Human IgG (positive control) | Thermo Fisher |
| Human Serum Albumin/HSA (negative control) | Sino Biological |
| 2019 SARS CoV2 Nucleocapsid (N) | Sino Biological |
| 2019 SARS CoV2 RBD (RBD) | Sino Biological |
| 2019 SARS CoV2 S1S2 + ECD (S1S2) | Sino Biological |
| 2019 SARS CoV2 S1 (S1) | Sino Biological |
| SARS CoV2 RBD - B.1.1.7 (N501Y) | Sino Biological |
| SARS CoV2 RBD - B.1.351 (K417N, E484K, N501Y) | Sino Biological |
| SARS CoV2 S1 - B.1.351 (K417N, E484K, N501Y, | Sino Biological |
| D614G) | |
| SARS CoV2 S1 - B.1.1.7 (ΔHV69-70, ΔY144, | Sino Biological |
| N501Y, A570D, D614G, P681H) | |

### Biological Samples

Serum samples were collected from COVID-19 patients admitted to Albany Medical Center between October and December 2020 who enrolled in a study “Defining genetic and immune factors in COVID-19 severity.” All patients had a positive RT-PCR test for CoV2 and were hospitalized due to severity of their illness. Sera were processed on the day of collection and placed into −80 C freezer until analyses. The study was approved by the IRB at Albany Medical Center (Protocol #5929).

Dried blood samples were collected by the finger stick method. Lancet devices (27 ga.) and Whatman 903 protein saver collection cards were sent to volunteers with instructions and consent form approved by the SUNY Polytechnic Institute Institutional Review Board (protocol #IRB-2020-10 and #IRB-2021-2). Blood droplets were collected, allowed to dry, and then either hand delivered or mailed (via US Postal Service) to SUNY Polytechnic Institute. Following receipt of DBS samples, a 6 mm diameter biopsy punch was used to remove samples from the collection cards, which were then soaked in 500 μl of PBS-T ˜12 hr at 4° C. with rocking. Samples were collected from 1) participants who had no known exposure to COVID-19 and/or tested negative for COVID-19 infection, 2) vaccinated individuals at different time points throughout vaccination sequence, including pre-vaccination, at the second dose, and two weeks after the second dose, 3) vaccinated individuals at a single time point, a minimum of 2 weeks after final vaccination dose, and 4) vaccinated individuals who were previously diagnosed with COVID-19 infection via RT-PCR. The age, gender, and exposure/vaccination information for all participants represented in this work are listed in supplementary tables S1 and S2. The age, gender, and exposure/vaccination information for all participants represented in this work are listed in tables 2 and 3.

TABLE 2 (Subject information for individuals who were infected with COVID-19 or vaccinated with the Pfizer-BioNTech, Moderna, or Johnson & Johnson vaccines. Blood serum samples from acutely infected individuals (hospitalized) were collected during hospitalization, while dried blood spots from infected (non-hospitalized) individuals were collected at least 4 weeks post recovery. For vaccinated individuals, dried blood spots samples were collected at least 2 weeks after the final dose of vaccine.)

TABLE 2

| | | | | | | Weeks Since | |
|---|---|---|---|---|---|---|---|
| | Vaccine/ | | | | Weeks past | Positive | |
| | Exposure Type | Sample # | Gender | Age | 2nd dose | Diagnosis | Sample Type |
| 1 | Pfizer | 1 | M | 54 | 2 | n/a | Dried Blood Spot |
| 2 | Pfizer | 2 | M | 43 | 2 | n/a | Dried Blood Spot |
| 3 | Pfizer | 3 | F | 54 | 2 | n/a | Dried Blood Spot |
| 4 | Pfizer | 4 | M | 57 | 2 | n/a | Dried Blood Spot |
| 5 | Pfizer | 5 | M | 34 | 8.5 | n/a | Dried Blood Spot |
| 6 | Pfizer | 6 | F | 38 | 4 | n/a | Dried Blood Spot |
| 7 | Pfizer | 7 | F | 42 | 2 | n/a | Dried Blood Spot |
| 8 | Pfizer | 8 | M | 72 | 2 | n/a | Dried Blood Spot |
| 9 | Pfizer | 9 | M | 61 | 2 | n/a | Dried Blood Spot |
| 10 | Pfizer | 10 | F | 66 | 2 | n/a | Dried Blood Spot |
| 11 | Pfizer | 11 | M | 67 | 2 | n/a | Dried Blood Spot |
| 12 | Pfizer | 12 | M | 37 | 4 | n/a | Dried Blood Spot |
| 13 | Pfizer | 13 | F | 43 | 4 | n/a | Dried Blood Spot |
| 14 | Pfizer | 14 | F | 64 | 2 | n/a | Dried Blood Spot |
| 15 | Pfizer | 15 | M | 73 | 3 | n/a | Dried Blood Spot |
| 16 | Pfizer | 16 | M | 24 | 2 | n/a | Dried Blood Spot |
| 17 | Pfizer | 17 | M | 44 | 2 | n/a | Dried Blood Spot |
| 18 | Moderna | 1 | F | 69 | 2 | n/a | Dried Blood Spot |
| 19 | Moderna | 2 | M | 69 | 2 | n/a | Dried Blood Spot |
| 20 | Moderna | 3 | F | 28 | 5 | n/a | Dried Blood Spot |
| 21 | Moderna | 4 | F | 57 | 2 | n/a | Dried Blood Spot |
| 22 | Moderna | 5 | F | 70 | 2 | n/a | Dried Blood Spot |
| 23 | Moderna | 6 | M | 53 | 2 | n/a | Dried Blood Spot |
| 24 | Moderna | 7 | F | 33 | 2 | n/a | Dried Blood Spot |
| 25 | Moderna | 8 | M | 38 | 2 | n/a | Dried Blood Spot |
| 26 | Johnson & Johnson | 1 | M | 65 | 6.5 | n/a | Dried Blood Spot |
| 27 | Johnson & Johnson | 2 | M | 42 | 2 | n/a | Dried Blood Spot |
| 28 | Johnson & Johnson | 3 | M | 24 | 2 | n/a | Dried Blood Spot |
| 29 | Johnson & Johnson | 4 | M | 29 | 2.5 | n/a | Dried Blood Spot |
| 30 | Johnson & Johnson | 5 | M | 58 | 4 | n/a | Dried Blood Spot |
| 31 | Johnson & Johnson | 6 | F | 19 | 2 | n/a | Dried Blood Spot |
| 32 | Johnson & Johnson | 7 | M | 34 | 3 | n/a | Dried Blood Spot |
| 33 | Johnson & Johnson | 8 | F | 19 | 4 | n/a | Dried Blood Spot |
| 34 | Johnson & Johnson | 9 | M | 33 | 4 | n/a | Dried Blood Spot |
| 35 | Previously Infected + Pfizer | 1 | M | 50 | 5 | 18 | Dried Blood Spot |
| 36 | Previously Infected + Pfizer | 2 | F | 49 | 6 | 18 | Dried Blood Spot |
| 37 | Previously Infected + Pfizer | 3 | M | 81 | 8 | 7.5 | Dried Blood Spot |
| 38 | Previously Infected + Moderna | 4 | F | 56 | 2 | 7.5 | Dried Blood Spot |
| 39 | Previously Infected + Pfizer | 5 | F | 18 | 2 | 22.5 | Dried Blood Spot |
| 40 | Previously Infected + Pfizer | 6 | F | 57 | 1.5 | 4.5 | Dried Blood Spot |
| 41 | Previously Infected + Pfizer | 7 | M | 55 | 2 | 9 | Dried Blood Spot |
| 42 | Previously Infected + Moderna | 8 | F | 18 | 3 | 20 | Dried Blood Spot |
| 43 | Previously Infected + Moderna | 9 | M | 68 | 4 | 54 | Dried Blood Spot |
| 44 | Previously Infected - non Hospitalized | 1 | M | 74 | n/a | 14 | Dried Blood Spot |
| 45 | Previously Infected - non Hospitalized | 2 | M | 66 | n/a | 5 | Dried Blood Spot |
| 46 | Previously Infected - non Hospitalized | 3 | M | 56 | n/a | 7 | Dried Blood Spot |
| 47 | Previously Infected - Hospitalized | AR7 | | | n/a | | Serum |
| 48 | Previously Infected - Hospitalized | AR21 | | | n/a | | Serum |
| 49 | Previously Infected - Hospitalized | GS12 | | | n/a | | Serum |
| 50 | Previously Infected - Hospitalized | GS14 | | | n/a | | Serum |
| 51 | Previously Infected - Hospitalized | GS17 | | | n/a | | Serum |

Total Male 28
Total Female 18

TABLE 3
(Subject information (Pfizer-BioNTech vaccinated). Dried blood
samples were collected just prior to the 1^{st} dose, at the time of the
2^{nd} dose, and 2 weeks after the 2^{nd} dose.)

| | Vaccine/ | | | | |
|---|---|---|---|---|---|
| | Exposure Type | Sample # | Gender | Age | Sample Type |
| 1 | Pfizer | 1 | M | 43 | Dried Blood Spot |
| 2 | Pfizer | 2 | M | 36 | Dried Blood Spot |
| 3 | Pfizer | 3 | F | 57 | Dried Blood Spot |
| 4 | Pfizer | 4 | F | 54 | Dried Blood Spot |
| 5 | Pfizer | 5 | M | 60 | Dried Blood Spot |
| 6 | Pfizer | 6 | F | 42 | Dried Blood Spot |
| 7 | Pfizer | 7 | M | 61 | Dried Blood Spot |
| 8 | Pfizer | 8 | M | 23 | Dried Blood Spot |
| 9 | Pfizer | 9 | F | 42 | Dried Blood Spot |
| 10 | Pfizer | 10 | M | 44 | Dried Blood Spot |

### GC-FP Detection Assay and ACE2 Competitive Assay

GC-FP microchips were processed using the same conditions described previously, See e.g., Cady, N.C., Tokranova, N., Minor, A., et al., Multiplexed detection and quantification of human antibody response to COVID-19 infection using a plasmon enhanced biosensor platform, Biosens Bioelectron, 171, 112679 (2021) (herein entirely incorporated by reference) (cited herein as Cady et al., 2021). Total assay time from sample introduction to chip imaging was 30 min. For serum testing, a standard dilution of serum in PBS-T (1:50) was used. For dried blood spot testing, undiluted extract (from extraction in 500 μl PBS-T) from the 6 mm diameter segment of the blood collection card was used in place of serum. Ciencia image analysis LabView software was used to define a region of interest (ROI) for each individual spot on the GC-FP biosensor chip and the fluorescence intensity of each spot was measured. The fluorescence intensity of all spots was normalized to the human IgG (Hum IgG) internal control spots on each chip, to account for variability between individual chips and individual experiments, generating a “GC-FP detection ratio” for every protein/antigen included in the GC-FP microchip (Cady et al., 2021):

$$GC \text{-} FP Detection Ratio = \frac{\overline{x} target spot intensity}{( \overline{x} neg . ctrl . spot intensity ) + ( 3 σ neg . ctrl . spot intensity )}$$

To determine if GC-FP antibody binding data for 2019 SARS CoV2 vs. B.1.1.7 and B.1.351 variant antigens was consistent with standard methods, an ELISA-based ACE2 competitive binding assay (Ray Biotech) was used, as per the manufacturer's instructions. Additional details are provided in the supplementary information. Percent binding inhibition was calculated for each sample by the following method:

$$\% Binding Inhibition = 1 - ( \frac{\overline{x} sample absorbance at 450 nm}{( \overline{x} positive control absorbance at 450 nm )} )$$

### Data Analysis

FP diagnostic ratio data and percent binding inhibition data for the ACE2 competitive assay were analyzed using GraphPad Prism 8.0 software (ROC analysis, correlation, and statistical analysis).

### Results and Discussion

### Determination of Human Antibody Response to 2019 SARS CoV-2 Vaccination

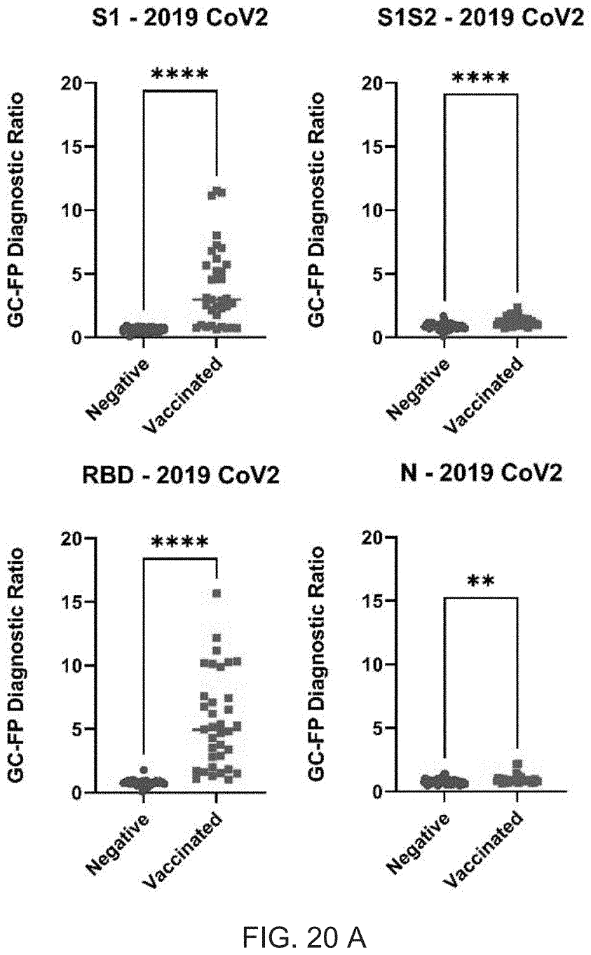

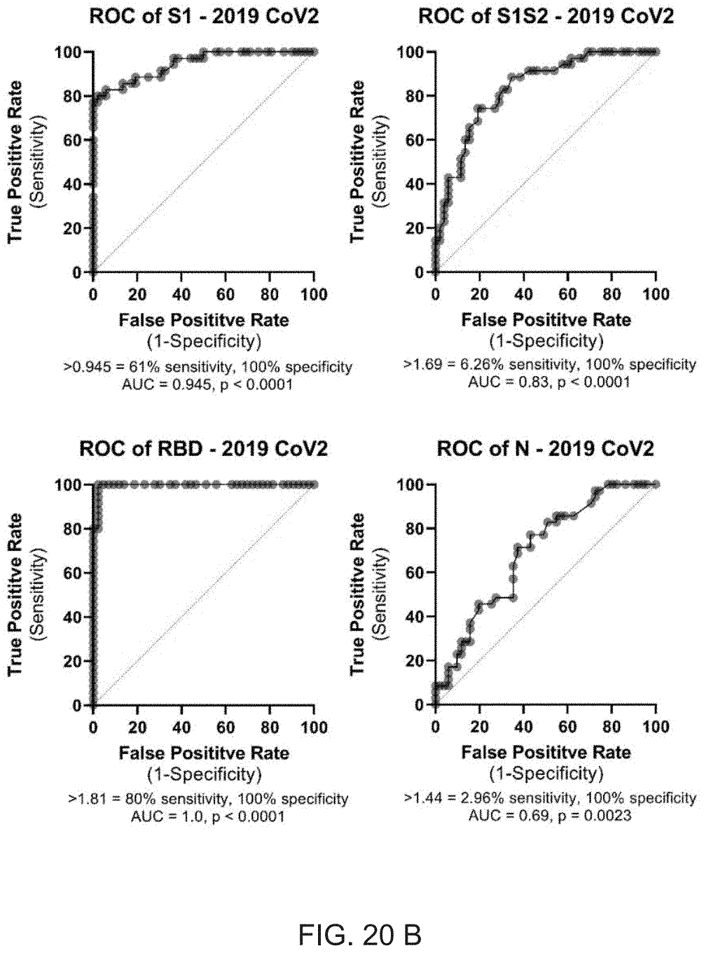

Dried blood samples from individuals with no known previous COVID-19 infection (n=52) and individuals two weeks past full vaccination with either Pfizer-BioNTech (n=17), Moderna (n=8), or Johnson & Johnson (n=9) vaccines were tested using the GC-FP assay to determine antibody levels against three SARS CoV2 spike protein antigens (S1, S1S2, RBD) and the nucleocapsid protein (N). As we described in previous work (Cady et al., 2021), the GC-FP diagnostic ratio provides a quantitative measure of antibody levels and correlates well with established serological techniques such as MIA and ELISA. In the current work, antibody levels in vaccinated individuals were significantly elevated for all spike antigens (Mann-Whitney, p<0.0001), with highest mean increase in antibody levels observed for the S1 and RBD antigens (SEE FIG. 20A). A nominal increase in antibody levels was also observed against the S1S2 antigen and N protein, but reactivity to these antigens was significantly less than for S1 and RBD. Receiver operator characteristic (ROC) analysis on these data (FIG. 20B) showed that antibody levels against S1 were diagnostic for vaccination status with 61% sensitivity and 100% specificity, while levels against RBD were diagnostic with 80% sensitivity and 100% specificity. Antibody levels for S1S2 and N resulted in low area under the curve (AUC) results from the ROC analysis and were considered insufficient for diagnostic purposes. Because all three vaccines should only elicit immunological response to the SARS CoV2 spike antigen, the lack of a diagnostic antibody response for the N protein was expected.

Referring to FIGS. 20A, and 20B, FIG. 20A depicts Human IgG levels against SARS CoV2 antigens, measured by GC-FP, for vaccinated individuals vs. unvaccinated individuals. The Mann-Whitney test and ROC analysis (see supplementary figure S2) were used to determine statistical significance (*p=0.03, ** p=0.002, *** p=0.0002, **** p<0.0001). FIG. 20B depicts ROC analysis of human IgG levels (measured by GC-FP) against 2019 SARS CoV2 for vaccinated vs. unvaccinated individuals. Area under the curve (AUC) and the GC-FP diagnostic ratio threshold are reported. The GC-FP diagnostic ratio threshold needed to achieve 100% specificity (false positive rate) is reported, as well as the sensitivity (true positive rate) that can be achieved while maintaining 100% specificity.

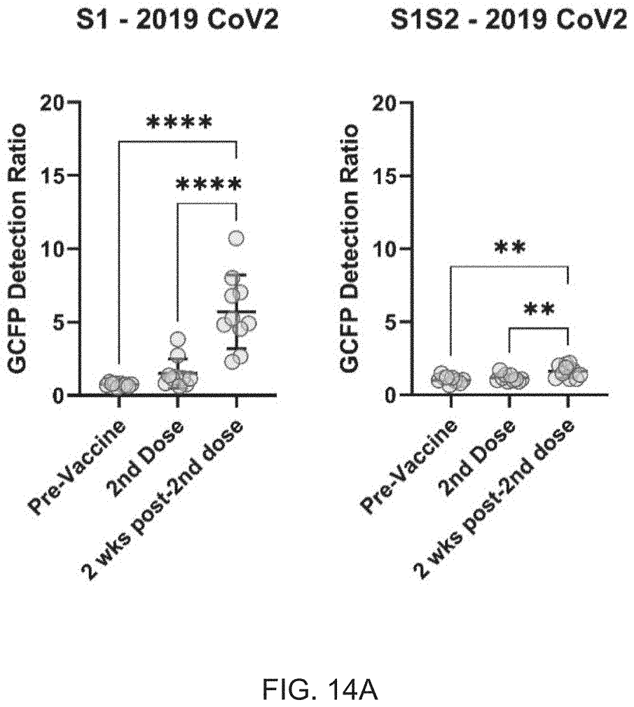

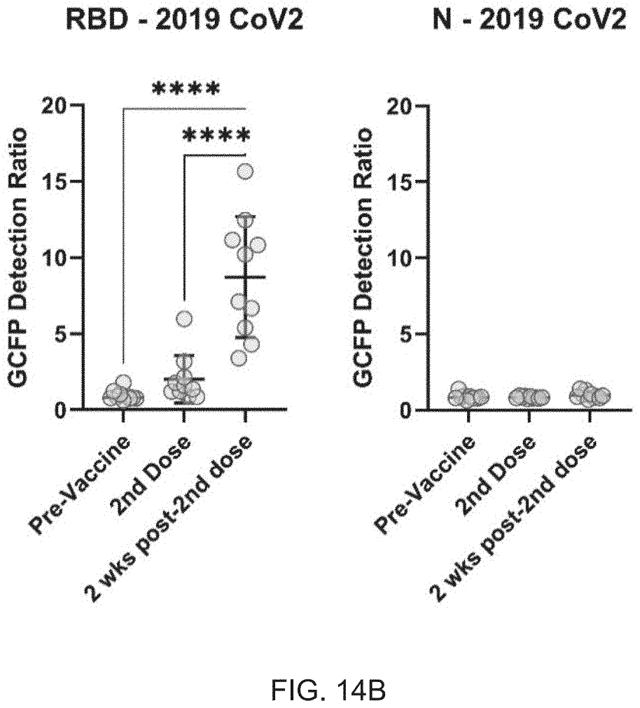

FIGS. 14A-14B depict human IgG levels against SARS CoV2 antigens throughout the vaccination sequence (Pfizer-BioNTech) for 10 different subjects, collected pre-vaccination, at the time of the 2^{nd} dose of vaccine, and 2 weeks after the 2^{nd} dose of vaccine. One-way ANOVA followed by Dunnett's multiple comparison testing was performed (*p=0.03, ** p=0.002, *** p=0.0002, **** p<0.0001).

Dried blood samples were also tested using GC-FP for ten (10) individuals throughout their vaccination sequence with the Pfizer-BioNTech vaccine (pre-vaccine, at the time of the 2^{st} dose, and 2 weeks after 2^{nd} dose). Antibody levels (as determined by GC-FP detection ratio) were significantly higher for the S1, S1S2, and RBD antigens at both the time of the 2^{nd} dose and 2 weeks after the second dose (FIG. 1). As expected, antibodies against the N antigen were not detected at any of these time points, since all three vaccines utilize the spike antigen (and not the N antigen) to induce immune response.

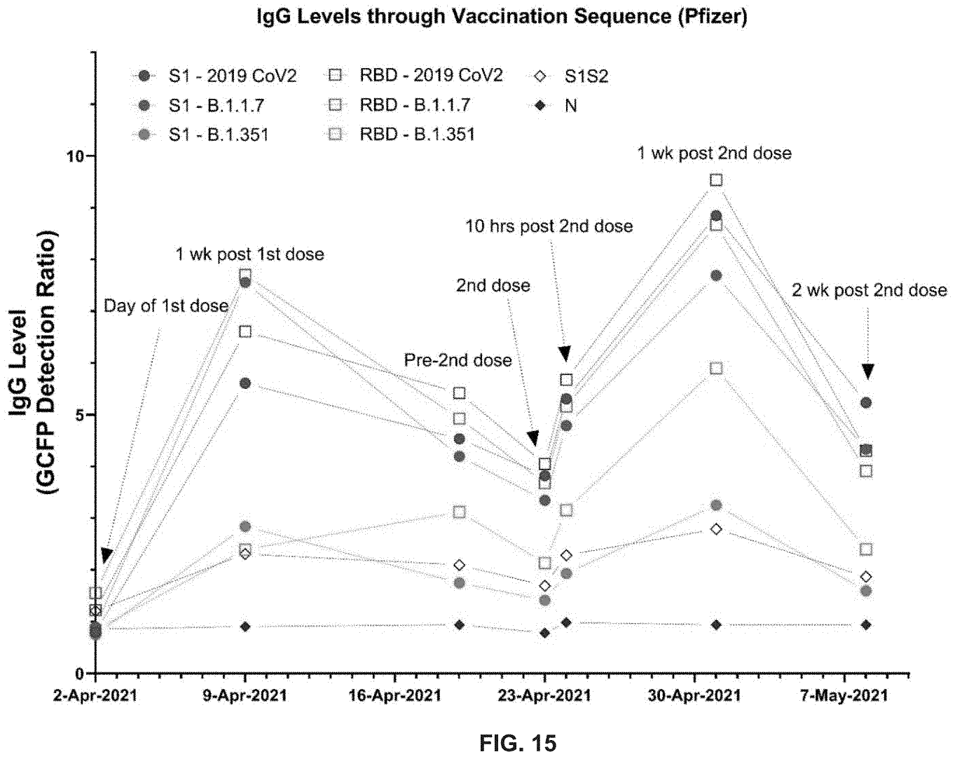

GC-FP was also used to detect antibody levels against SARS CoV2 antigens and antigens from variants B.1.1.7 and B.1.351, at additional time points throughout the vaccination sequence (Pfizer-BioNTech) for an individual subject (FIG. 15). Increased antibody levels were observed against spike antigens (S1, S1S2, and RBD) within 1 week after the 1st dose. Antibody levels then declined slightly until the 2^{nd} dose, when they increased at both 10 hrs and 1 week post 2^{nd} dose. Finally, levels declined at 2 weeks post 2^{nd} dose. The changes in antibody levels correlate well with what is expected during the vaccination sequence, wherein antibody levels should increase after each dose, but then decline to a stable level over time. Antibody responses to S1 or RBD from CoV2 UK variants were similar as antigens from the original 2019 CoV2, but these responses were dramatically reduced to S. African variants (RBD and S1 from strain B.1.351) throughout the vaccination course. These data illustrate the potential to quantitatively measure antibody response to vaccination with high resolution (through time). FIG. 15 depicts Human IgG levels for a single individual over the course of vaccination with the Pfizer-BioNTech vaccine, measured with GC-FP.

### Determination of Human Antibody Binding to 2019 SARS CoV-2 Antigens and its Variants

GC-FP testing was performed on serum from acutely infected (hospitalized) individuals and dried blood samples from individuals who were: 1) Uninfected/pre-vaccine—had no known prior infection with COVID-19 and were not vaccinated, 2) Hospitalized—infected with COVID-19 and hospitalized due to infection, 3) Non-hospitalized—were infected with COVID-19 and at least 4 weeks post recovery, 4) Vaccinated (Pfizer, Moderna, J&J—were at least 2 weeks past final dose of Pfizer-BioNTech, Moderna or Johnson & Johnson vaccine, or 5) CoV2 positive & Vaccinated—previously infected with COVID-19 and then fully vaccinated with Pfizer-BioNTech or Moderna vaccine. Results of this study are shown in FIG. 3. As compared to uninfected/unvaccinated individuals, vaccinated individuals (with and without prior infection) had significantly higher antibody levels against S1 and RBD antigens. Acutely infected (hospitalized) individuals had elevated antibody levels for S1S2 and N antigens, while non-hospitalized patients only showed increased antibody levels against N. Notably, individuals who were vaccinated after prior infection had the highest mean antibody levels against S1 and RBD. This is consistent with the fact that these individuals were effectively exposed to the spike antigen at three different time points (during infection and at the 1st and 2^{nd} doses of vaccine).

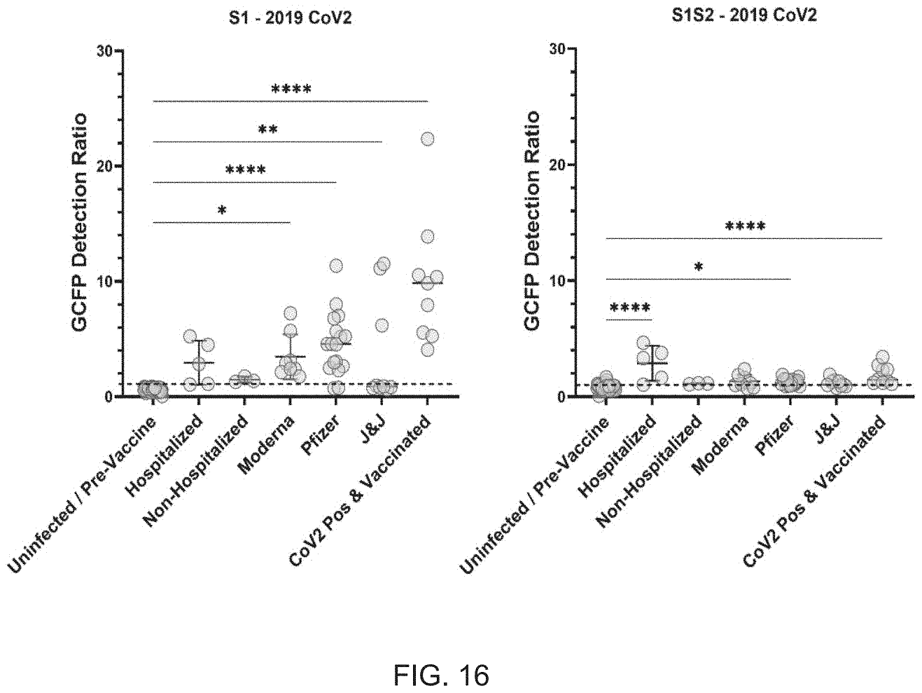

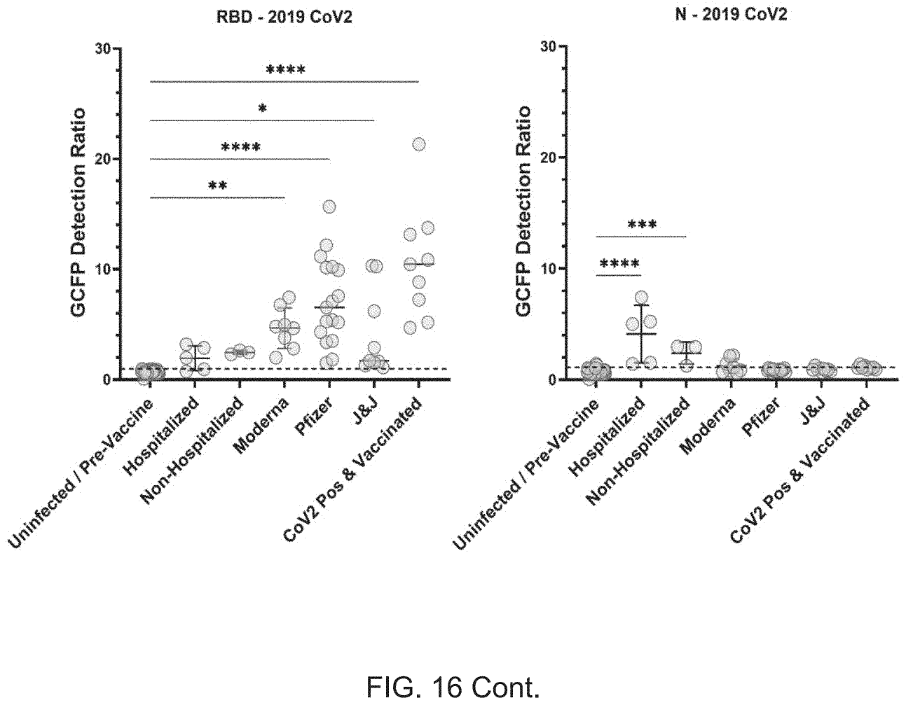

FIG. 16 depicts IgG levels against SARS CoV2 antigens for uninfected, previously infected, and vaccinated individuals. Uninfected samples were collected prior to vaccination, from individuals who reported no prior COVID-19 symptoms, and tested negative via PCR and/or antibody testing (n=42). Other samples were from PCR confirmed COVID-19 positive subjects who were hospitalized (n=3), PCR confirmed COVID-19 positive subjects who were not hospitalized CoV2 (n=5), and previously COVID-19 positive subjects who received subsequent vaccination (n=9). Samples were also collected from subjects who were at least 2 weeks past full vaccination with Pfizer-BioNTech (n=17), Moderna (n=8), or 2 weeks after receiving the Johnson & Johnson vaccine (n=9). One-way ANOVA followed by Dunnett's multiple comparison testing was performed (*p=0.03, ** p=0.002, *** p=0.0002, **** p<0.0001).

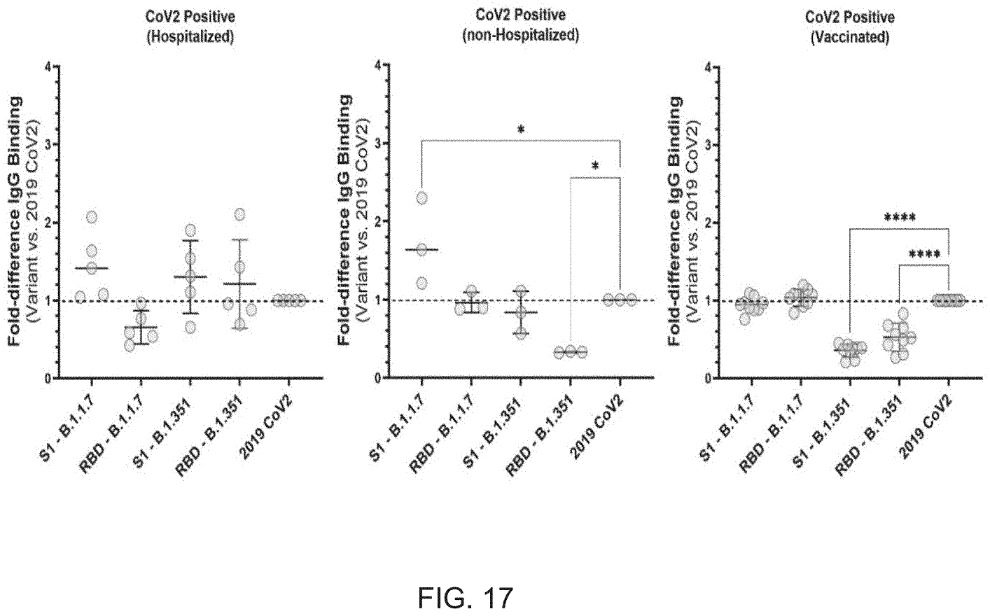

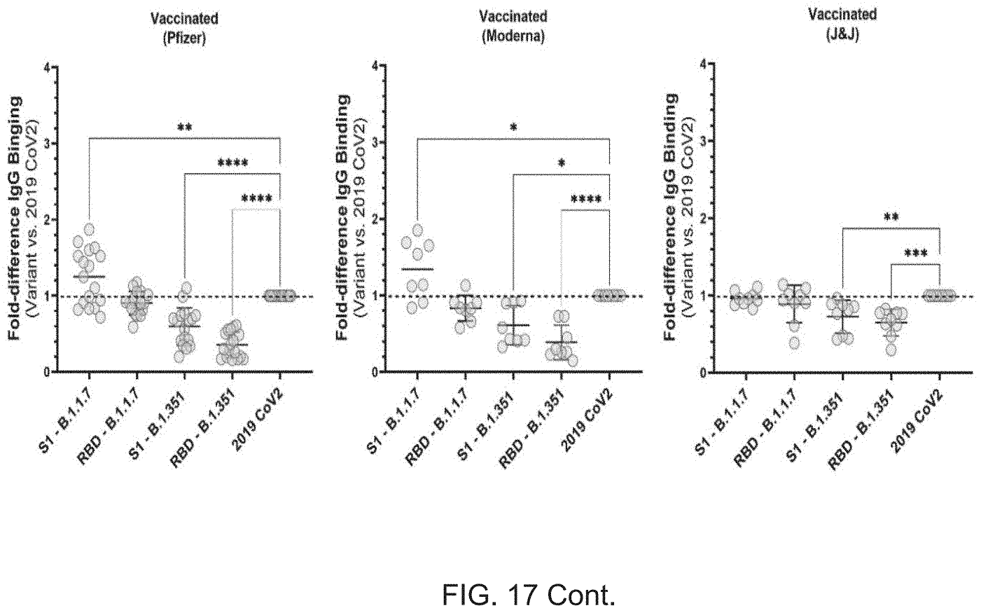

Between individual subjects, antibody levels were highly variable, regardless of the mode of COVID-19 exposure or vaccination status (FIG. 3 and FIG. 10). To account for these differences, and to elucidate relative antibody binding levels to variant antigens vs. 2019 SARS CoV2 antigens, the fold-difference (binding to variant antigen vs. 2019 SARS CoV2 antigen) in antibody levels between variant antigens and the 2019 SARS CoV2 antigens was plotted (FIG. 17). For all vaccinated individuals, antibody binding to B.1.351 antigens (both RBD and S1) was reduced vs. 2019 SARS CoV2 antigens. Antibody binding to B.1.1.7 antigens was either equivalent to, or slightly higher than 2019 SARS CoV2 antigens for these same individuals. For previously infected individuals (hospitalized and non-hospitalized) there were only minor differences in antibody levels for variant antigens vs. 2019 SARS CoV2 antigens (p>0.03 or not significant).

FIG. 17 depicts fold-difference in antibody levels against antigens from SARS CoV2 variant strains B.1.1.7 and B.1.351 vs. antigens from the original 2019 SARS CoV2 strain. Samples included those from individuals who were hospitalized (n=3); non-hospitalized CoV2 positive (n=5); previously CoV2 positive with subsequent vaccination (n=9); and at least 2 weeks past vaccination with Pfizer-BioNTech (n=17), Moderna (n=8), or Johnson & Johnson vaccine (n=9). One-way ANOVA followed by Dunnett's multiple comparison testing was performed (*p=0.03, ** p=0.002, *** p=0.0002, **** p<0.0001).

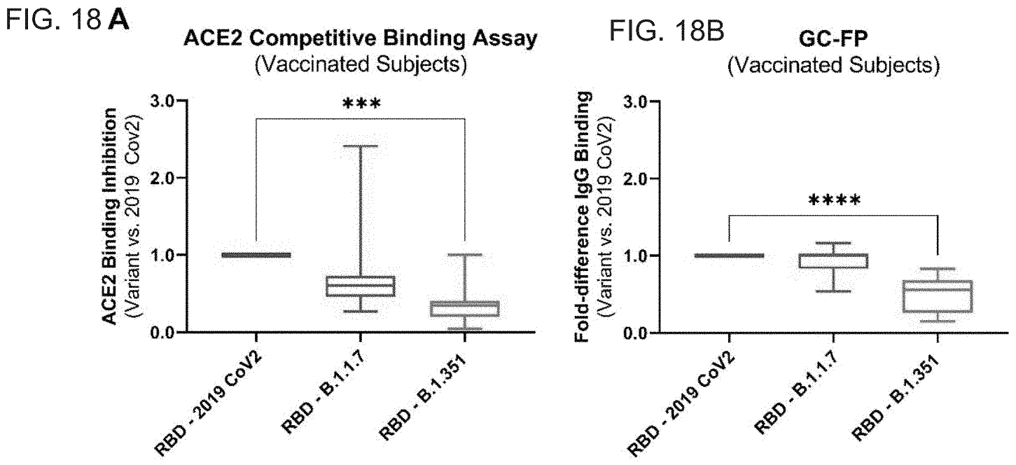

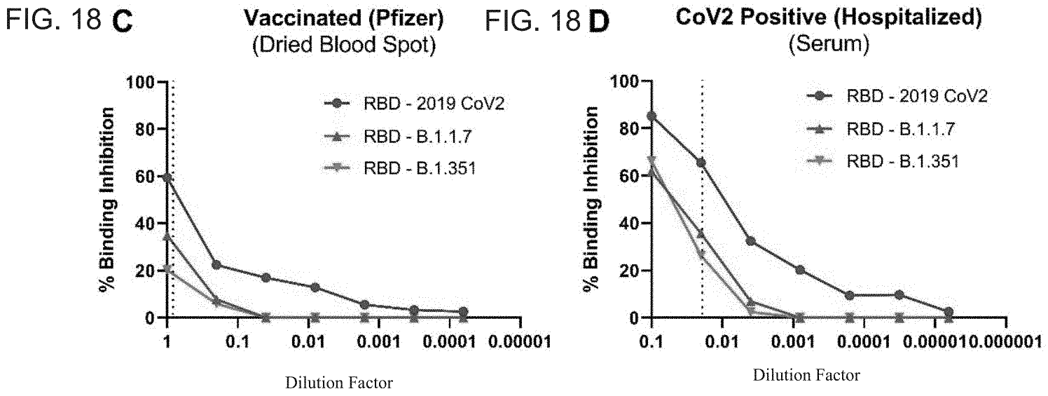

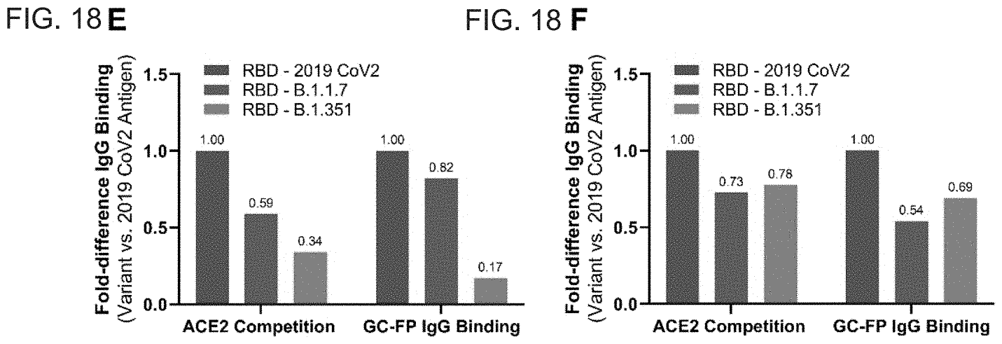

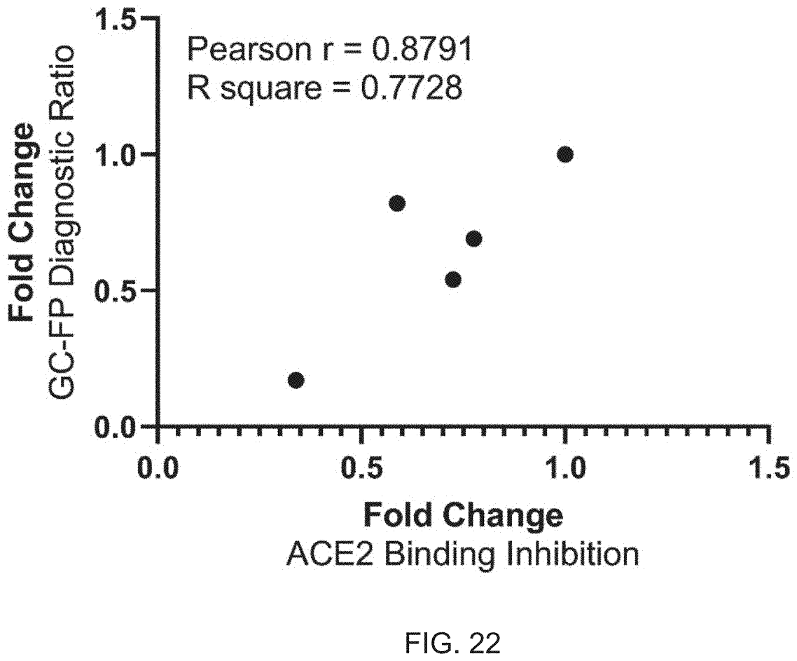

To determine if these results were due to differences in antibody affinity towards variant vs. 2019 SARS CoV2 antigens, we compared the GC-FP results to a competitive ELISA in which blood samples were mixed with ACE2 receptor protein (the cellular target of SARS CoV2) and allowed to competitively bind to 2019 SARS CoV2 RBD antigen, or the B.1.1.7 and B.1.351 RBD variants. Competitive ELISA results were similar to GC-FP results (FIGS. 18A and 18B), showing both significantly reduced antibody binding (GC-FP) and reduced binding inhibition (competitive ELISA) for the B.1.351 RBD variant. When quantitative dilution testing was performed for a single Pfizer-BioNTech-vaccinated individual and a single acutely infected (hospitalized) individual, significant differences in binding inhibition were observed for both B.1.1.7 and B.1.351 RBD variants (FIGS. 18C and 18D). When the fold-difference in antibody binding (GC-FP) and percent binding inhibition (competitive ELISA) were compared, there was close correlation between the two methods (FIGS. 18E and 18F, and FIG. 22). Values plotted in FIGS. 18E and 18 were shown to be correlated (Pearson r=0.89, p=0.02, FIG. 22), suggesting that GC-FP has utility for assessing relative antibody binding levels and/or avidity to antigens from 2019 SARS CoV2 and variant strains of the virus.

FIGS. 18A and 18B) IgG IgG levels from dried blood spots measured by GC-FP diagnostic ratio compared to competitive ELISA by eluate from the same dried blood spot samples. Both GC-FP and ACE2 competitive binding were performed for RBD antigen from the original 2019 SARS CoV2 and the variant strains B.1.1.7 and B.1.351. Testing was performed with dried blood spots collected from vaccinated subjects (3 Pfizer-BioNTech, 3 Moderna, 2 Johnson & Johnson) and subjects who were both previously infected and then vaccinated with Pfizer-BioNTech or Moderna vaccines (n=3). One-way ANOVA followed by Dunnett's multiple comparison testing was performed (*p=0.03, ** p=0.002, *** p=0.0002, **** p<0.0001). Percent ACE2 binding inhibition from competitive ELISA assay is shown for a dried blood spot sample (FIG. 18C) from a Pfizer-BioNTech vaccinated subject and blood serum from a hospitalized, COVID-positive subject (FIG. 18D). Vertical dotted lines represent the dilution factor used in the corresponding GC-FP test for each sample. Fold difference in binding inhibition and fold difference in GC-FP diagnostic ratio was plotted for variant antigens (RBD B.1.1.7 and RBD B.1.351) vs. RBD 2019 CoV2 (FIGS. 18E &18F).

ACE2 Competitive Binding Assay Method:

Briefly, 2019 SARS CoV2 RBD, B.1.1.7 RBD, or B.1.351 RBD variants were pre-coated in microtiter plate wells (as received). Diluted blood serum and dilutions of PBS-T eluted dried blood spot samples were then mixed with ACE2 protein and distributed to individual wells. In one study, serum and eluted sample from dried blood samples were serially dilute in a 1:5 dilution series and then subjected to the competitive assay. In the second study, serum and eluted sample from dried blood samples were diluted to the same concentration used for GC-FP and then subjected to the competitive assay. Competitive assay plates were incubated overnight at 4° C. after addition of sample and ACE2 protein. Wells were then washed 4× with 300 μl Ray Biotech wash solution, followed by introduction of an anti-ACE2 labeling antibody. After incubation at RT for 1 hr, wells were again washed 4× with wash solution, followed by introduction of HRP-labeled secondary antibody and incubation for an additional 1 hr. After a final 4× wash step, colorimetric reagent was added and plates were incubated for 30 min at room temp, followed by addition of stop solution and absorbance reading at 450 nm.

Referring now to FIG. 19, a GC-FP SARS CoV2 antigen chip suitable for use in accordance with the present disclosure is shown. The table on the right correlates that antigens on the chip with the spot number.

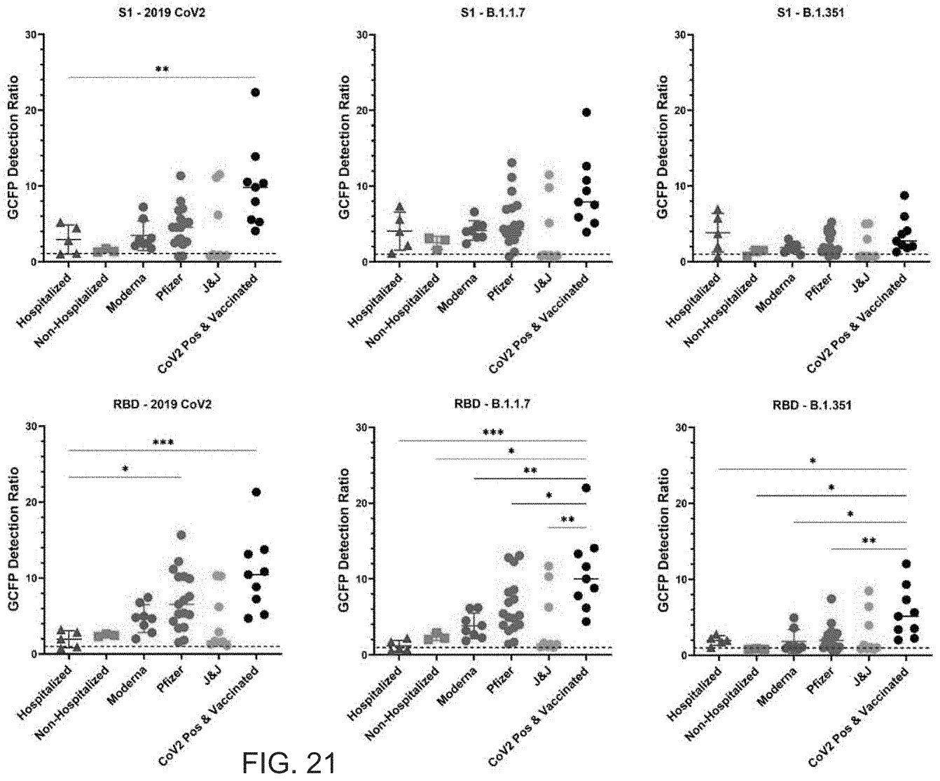

Referring now to FIG. 21, Human IgG levels against SARS CoV2 antigens from the original 2019 SARS CoV2 strain and variant strains B.1.1.7 and B.1.351. GC-FP diagnostic ratio is reported as a measure of IgG level against each antigen, for multiple exposure scenarios and vaccination status (hospitalized and non-hospitalized CoV2 positive, previously CoV2 positive with subsequent vaccination, and at least 2 weeks past vaccination with Pfizer-BioNTech, Moderna or Johnson & Johnson vaccine). One-way ANOVA followed by Dunnett's multiple comparison testing was performed (*p=0.03, ** p=0.002, *** p=0.0002, **** p<0.0001).

Referring to FIG. 22 a plot depicts the correlation of the fold change in ACE2 binding inhibition or GC-FP diagnostic ratio for B.1.1.7 and B.1.351 variants of RBD vs. 2019 CoV2 RBD.

### CONCLUSIONS

GC-FP is a rapid and accurate technique for detection of antibodies that result from COVID-19 infection (Cady et al.; 2021). In the current study, we extended this work to show that GC-FP can measure the level of antibodies resulting from vaccination, and that it can quantitatively measure the increase in antibody levels during the course of vaccination for multiple target antigens. Using ROC analysis, GC-FP diagnostic ratio thresholds could be established to yield a clear cut-off for determining whether or not an individual has been vaccinated. When using the RBD antigen, this results in 80% sensitivity and 100% specificity for all three of the currently approved vaccines (Pfizer-BioNTech, Moderna and Johnson & Johnson). Furthermore, our results show that antibody levels can be measured with high resolution throughout the course of vaccination, making it a useful tool to track the progression of an individual's serological response to a vaccine. Highlighting the sensitivity of the GC-FP approach, we were able, to detect increasing levels of antibodies within just 10 hrs of an individual's second dose of the Pfizer-BioNTech vaccine (FIG. 15).

Beyond determination of vaccination status and serological response to vaccination, the multiplexed nature of GC-FP makes it highly amenable to measuring antibody binding to multiple antigens from both the original 2019 SARS CoV2 virus and the emerging variants of this virus. This is extremely important as variants of the virus continue to emerge (See e.g., Mahase, E., 2021. BMJ 372, n158, and Wise, J., 2021. BMJ 373, n1315). For example, GC-FP results indicate reduced antibody binding to the RBD and S1 antigens of the B.1.351 (S. African) variant, which was further confirmed through competitive ELISA-based testing (FIGS. 18A-18F). When individual samples were evaluated by GC-FP and competitive ELBA, reduced antibody binding to antigens from both the B.1.1.7 (UK) and B.1.351 variants was observed.

The results of the present study, together with our previous demonstration of GC-FP for COVID-19 antibody detection (Cady et al., 2021) compare favorably to similar studies using multiplexed, array-based techniques. Using a plasmonic-based approach, Liu et al. demonstrated high throughput detection of COVID-19 antibodies from human serum and saliva (See e.g., Liu, T., Hsiung, J., Zhao, S., et al., 2020. Nat Biomed Eng 4(12), 1188-1196). This approach requires significantly more time than GC-FP (˜2 hrs vs. 30 min) but it has the advantage of measuring the relative avidity of antibodies towards target antigens. In another study, Swank et al., demonstrated an elegant, high throughput microfluidic approach to COVID-19 antibody detection. While this approach enables extremely high throughput, it is limited in the number of target antigens that can be implemented, and requires complicated printing of human blood samples to prepare detection chips.

Here, it is demonstrated that GC-FP based, multiplexed detection of antibody binding from human blood is a useful tool for determining an individual's response to vaccination and the relative binding of antibodies to variants of the 2019 SARS CoV2 virus. When considering the rapid time to result (30 min) for the GC-FP assay and the ability to target a large number of antigens at once, this represents a powerful tool for continued management of the global COVID-19 pandemic, and with broad applicability to other diseases and vaccines.

### Example 2

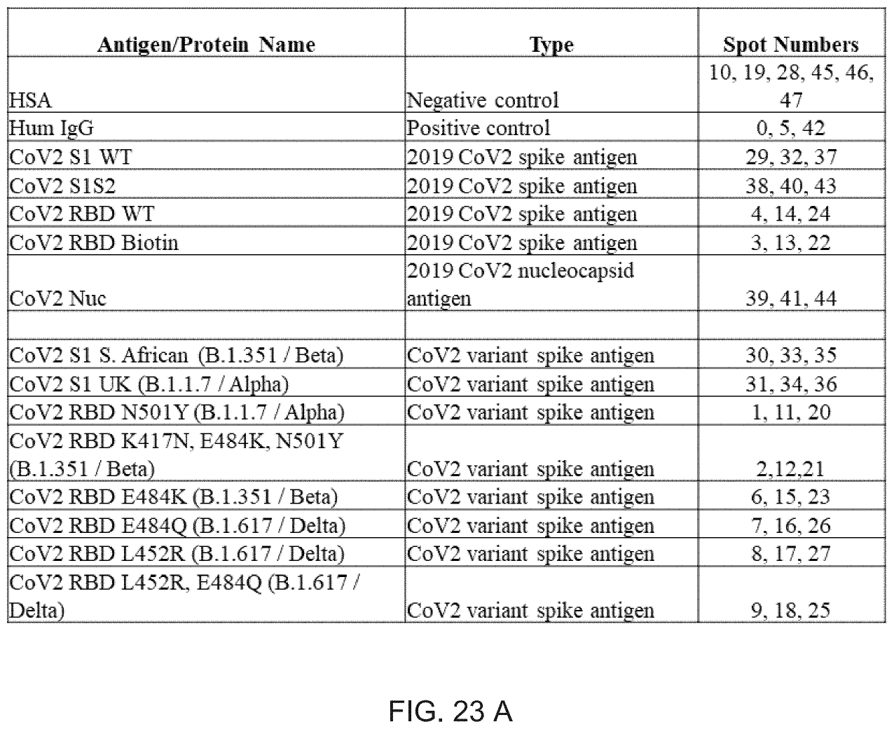

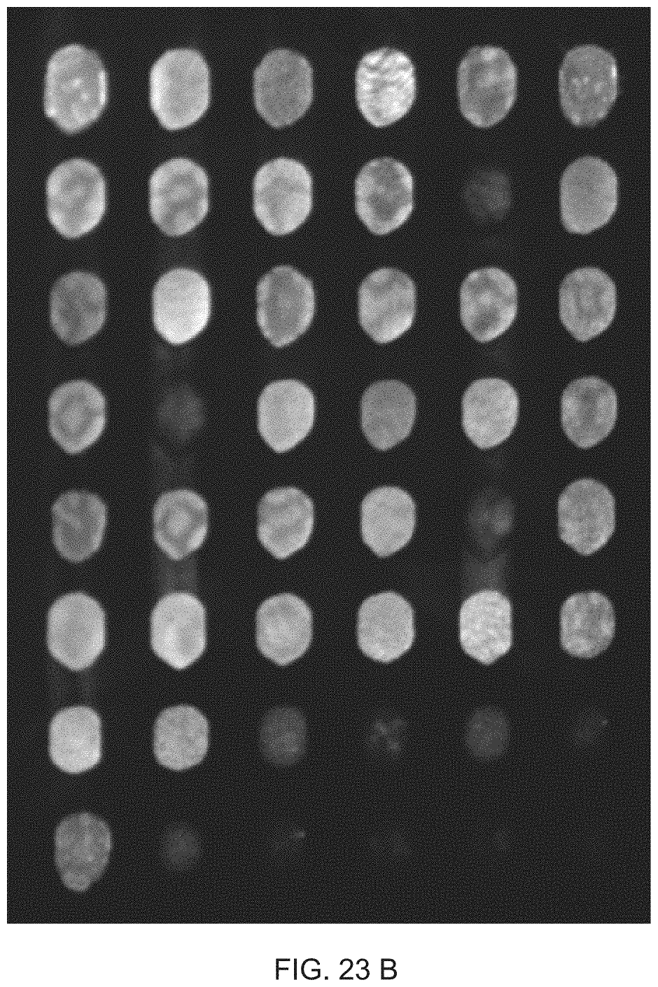

FIG. 23B depicts a top-down view of an antigen chip of the present disclosure, and FIG. 23A depicts the antigen and spot number on the chip including a plurality of antigens to a plurality of SARS-CoV-2 variants. More specifically the photograph depicts a chip provided includes 2 SARS-CoV-2 variants of the S1 region of the spike protein and 6 variants of SARS-CoV-2 of the RBD region of the spike protein. In embodiments the RBD variants cover all three major variants (currently)—B.1.1.7/Alpha; B.1.351 Beta, B.1.617/Delta). The image in FIG. 23B shows the IgG binding profile to these antisense for an individual or subject who was previously positive for COVID-19 or SARS-CoV-2 infection.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed is:

1. A method for determining a humoral response due to a presence of a target infectious agent or vaccine, comprising:
capturing one or more biomarkers on a substrate that binds a plurality of biomarkers to one or more binding sites, wherein one or more binding sites bind one or more biomarkers, and wherein when one or more biomarkers are present, forming one or more bound biomarkers-of-interest;
contacting the one or more bound biomarkers-of-interest with one or more fluorescent binding partners to form one or more fluorescent complexes;
contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes;
detecting emission light of the one or more fluorescent complexes; and
determining a type and/or quantity of the plurality of biomarkers.

2. The method of claim 1, wherein the one or more biomarkers are immunoglobulins.

3. The method of claim 2, wherein the immunoglobulins comprise one or more of immunoglobulin G (IgG), immunoglobulin M (IgM), immunoglobulin A (IgA), or isotypes or combinations thereof.

4. The method of claim 3, wherein the immunoglobulins comprise one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.

5. The method of claim 1, wherein the one or more biomarkers are serum antibody targets.

6. The method of claim 1, wherein the substrate is disposed within a cell.

7. The method of claim 6, wherein the cell is a fluidic flow cell or a test-tube.

8. The method of claim 1, wherein each binding site of the plurality of binding sites comprises two or more predetermined antigens configured to bind to two or more different predetermined biomarkers-of-interest.

9. The method of claim 1, wherein the plurality of biomarkers are derived from blood serum, blood plasma, whole blood, dried blood.

10. The method of claim 1, wherein the type is characterized as immunoglobulin G (IgG), immunoglobulin M (IgM), immunoglobulin A (IgA), or isotypes or combinations thereof.

11. The method of claim 1, wherein the type is further characterized as virus specific to COVID-19, SARS-CoV-2, or a variant of SARS-CoV-2.

12. The method of claim 1, wherein the plurality of biomarkers are antibodies specific to a SARS-COV-2 full-length spike protein (SEQ ID NO:1), SARS-COV-2 spike 1 protein (SEQ ID NO: 2), SARS-COV-2 envelope protein (N) (SEQ ID NO: 3).

13. The method of claim 1, wherein contacting the substrate with a source of collimated, polarized light, further comprises positioning the substrate within a grating-coupled fluorescent plasmonic (GC-FP) detection platform.

14. The method of claim 1, wherein the source of collimated polarized light is configured to cause fluorescent emission intensity greater than 10×, greater than 100×, greater than 500×, or greater than 1000×.

15. The method of claim 1, wherein detecting emission light of the one or more fluorescent complexes further comprises forming a fluorescent image on an antigen array or map of antigens upon the substrate.

16. The method of claim 1, wherein detecting emission light of the one or more fluorescent complexes further comprising normalizing a fluorescence intensity of a plurality of spots to a control spot.

17. The method of claim 16, wherein normalizing further comprises generating a GC-FP detection ratio.

18. An apparatus for quantitative analysis of a plurality of biomarkers contained in a sample, comprising:
a substrate comprising a plurality of binding sites, one or more of the plurality of binding sites comprises one or more predetermined antigens configured to bind to one or more predetermined biomarkers-of-interest and form one or more bound biomarkers-of-interest when biomarkers-of-interest are flowed over the substrate, and wherein the biomarkers-of-interest, when present, are bind one or more fluorescent binding partners to form one or more fluorescent complexes; and
a grating-coupled fluorescent plasmonic (GC-FP) detection platform, wherein the platform contacts the substrate with a source of collimated, polarized light, wherein the source of collimated polarized light emits light at a predetermined wavelength appropriate for transferring energy to a plurality of surface plasmons and excite fluorescence of the one or more fluorescent complexes, wherein the one or more biomarkers-of-interest are selected from immunoglobulins comprising one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.

19. The apparatus of claim 18, wherein the apparatus detects and normalizes fluorescence of the one or more fluorescent complexes.

20. A method for determining a humoral response due to a presence of a target infectious agent or vaccine, comprising:
detecting in a biological sample of a subject at least one target antibody that is susceptible of being produced by the subject when the subject is vaccinated or infected by a target infectious agent, wherein the method comprises:
capturing the at least one target antibody on a substrate that binds the at least one target antibody to a plurality of binding sites, wherein one or more binding sites bind one or more target antibodies, and wherein when one or more target antibodies are present forming one or more bound biomarkers-of-interest;
contacting the one or more bound biomarkers-of-interest with one or more fluorescent binding partners to form one or more fluorescent complexes;
contacting the substrate with a source of collimated, polarized light, wherein the source of collimated polarized light emits light at a predetermined wavelength appropriate for transferring energy to a plurality of surface plasmons and excite fluorescence of the one or more fluorescent complexes;
detecting emission light of the one or more fluorescent complexes; and
determining a type and/or quantity of the at least one target antibody, wherein the at least one target antibody is selected from immunoglobulins comprising one or more of immunoglobulin G (IgG) directed against SARS-CoV-2, a SARS-CoV-2 variant, and/or a COVID 19 vaccine, immunoglobulin M (IgM) directed against SARS-CoV-2, a SARS-CoV-2 variant, and/or a COVID 19 vaccine, immunoglobulin A (IgA) directed against SARS-CoV-2, a SARS-CoV-2 variant, and/or a COVID 19 vaccine, or isotypes or combinations thereof.
